(19) United States
(12) Patent Application Publication
(10) Pub. No.: US 2024/0327857 A1
(43) Pub. Date: Oct. 3, 2024

(54) INCREASING SEED LIPID CONTENT BY OVER-EXPRESSING TRANSCRIPTIONAL REGULATORS

(71) Applicant: Purdue Research Foundation, West Lafayette, IN (US)

(72) Inventors: Kranthi Varala, West Lafayette, IN (US); Ying Li, West Lafayette, IN (US); Rajeev Ranjan, West Lafayette, IN (US)

(73) Assignee: Purdue Research Foundation, West Lafayette, IN (US)

(21) Appl. No.: 18/593,007

(22) Filed: Mar. 1, 2024

Related U.S. Application Data
(60) Provisional application No. 63/487,763, filed on Mar. 1, 2023.

(51) Int. Cl. C12N 15/82; A01H 6/20

(52) U.S. Cl. CPC C12N 15/8247; A01H 6/20; C12N 15/8223

(57) ABSTRACT

Expression cassettes, plant cells, plant seeds, plants, and methods for modulating lipid content of seeds and growth of such plants. The expression cassette comprises a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

### CROSS REFERENCE TO RELATED APPLICATION

This application claims priority to U.S. provisional patent application No. 63/487,763, which was filed Mar. 1, 2023. The entire contents of the provisional patent application and the appendix thereto are hereby incorporated by reference in their entireties.

### STATEMENT OF GOVERNMENT SUPPORT

This invention was made with government support under grant numbers DE-SC0020399 and DE-SC001437 awarded by the Department of Energy. The government has certain rights in the invention.

### SEQUENCE LISTINGS

The sequences herein (SEQ ID NOS: 1-20) are also provided in computer-readable form encoded in an electronic file submitted herewith. The contents of the electronic sequence listing (70138-02_SequenceListing_27Feb2024.xml; size 36 KB; created Feb. 27, 2024) are incorporated herein by reference. The information recorded in computer-readable form is identical to the written sequence listings provided herein, pursuant to 37 C.F.R. § 1.821 (f).

### BACKGROUND

Seeds are the primary storage organs for lipids in plants. Seed lipids have a high energy/mass ratio and serve as an energy reserve for early growth of seedlings. Additionally, plant seeds store carbohydrates in the form of starches and proteins. The relative ratio of lipids to starches to proteins is highly variable from one plant species to another and can differ significantly between varieties in the same species. Plant species that naturally have a high lipid to starch ratio have been domesticated and cultivated by humans as a source of lipids for nutrition, energy and industrial purposes. Examples of plant species that currently serve as lipid sources include corn, soybean, sunflower, and peanuts.

In view of the above, it is an object of the present disclosure to provide materials and methods for modulating (such as increasing or decreasing) the lipid content of seeds. This and other objects and advantages, as well as inventive features, will be apparent from the description provided herein.

### SUMMARY

Described herein are plant cells, plant seeds, plants, and methods for improving or reducing lipid content of seeds and cultivating plants, plant cells, and plant seeds to obtain seeds with modulated (such as increased or decreased) lipid content. The nucleic acids, expression cassettes, plants, plant cells, seeds and methods described herein can be used to improve or reduce the lipid content of seeds in plants, such as oil crops, for human nutrition, biofuels, animal feedstock, cosmetics, industrial chemicals, and other uses. Methods of cultivating such plant seeds, plant cells, and plants include, for example, harvesting the plants, seeds, or the tissues of the plants. Such methods can also include isolating the lipids or starches from the plant seeds, plant cells, or plants.

For example, described herein is a plant cell, plant seed, or plant comprising an expression system comprising an expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof. Also described herein is an expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

In addition, methods are described herein for growing a plant comprising (i) introducing into at least one plant cell at least one transgene or expression cassette encoding a polypeptide for MYBS2, bHLH093, ATHB25, DiV2, CESTA1, TGA4, SPL12, or AGL18, or a combination of two, three, four, five, six, seven or all thereof, to generate one or more transformed plant cells and (i) generating a plant from the one or more transformed plant cell(s).

### DRAWINGS

FIG. 1 is a graph depicting total fatty acid content of Arabidopsis thaliana seeds expressing mutant lipid-regulating transcription factors with fatty acid content measured as fatty acid methyl ester (FAME) in g/mg of seeds. An unmutated line (Col-0) serves as the reference for seed lipid content in Arabidopsis thaliana.

FIG. 2 is a graph depicting total fatty acid content of Arabidopsis thaliana seeds from either control line (Col-0) or lines over-expressing lipid-regulating transcription factors with fatty acid content measured as FAME in g/mg of seeds.

FIGS. 3A-B are graphs showing mutation of the MYBS2 lipid-regulating transcription factor reduces fatty acid content of Arabidopsis thaliana seeds. FIG. 3A is a graph depicting total fatty acid content of two independent genetic lines of Arabidopsis thaliana expressing mutant MYBS2 lipid-regulating transcription factor, with the fatty acid content measured as FAME in g/mg of seeds. FIG. 3B is a graph depicting the fatty acid composition of Arabidopsis thaliana seeds expressing the mutant MYBS2 lipid-regulating transcription factor.

FIG. 4 is a schematic of the locations and relative positions of gene elements of the pGWB614 plant expression vector comprising a 35S promoter for inducing expression of a lipid-regulating transcription factor coding sequence (TF CDS).

FIG. 5 is a table showing a summary of seed lipid content changes caused by altering expression of lipid-regulating transcription factors.

FIGS. 6A-F show over-expression of transcription factors causes an increase in total oil accumulation in Arabidopsis seeds. Total oil content (measured as FAME/mg seeds) in the independent overexpression lines of transcription factors CESTA (FIG. 6A), bHLH93 (FIG. 6B), HB25 (FIG. 6C), MYBS2 (FIG. 6D), SPL12 (FIG. 6E), and AGL18 (FIG. 6F) along with control untransformed plants. The percentage increase in the total oil content compared to the corresponding untransformed plants is shown with blue numbers.

### DETAILED DESCRIPTION

Described herein are expression cassettes, plant cells, plant seeds, plants, and methods useful for improving or reducing the lipid content of plant seeds. The plant cells, plant seeds, and plants can include an expression cassette encoding a nucleic acid segment encoding a lipid-regulating transcription factor polypeptide or inactivating mutants thereof. For example, the lipid-regulating transcription factor can include MYBS2, bHLH093, ATHB25, DiV2, CESTA1, TGA4, SPL12, or AGL18, or a combination thereof, such as any two, three, four, five, six, seven or all eight transcription factors. Increased expression of one or more of the lipid-regulating transcription factors can increase the lipid content of plant seeds by approximately 10% to approximately 20% compared to plant seeds that do not have the expression cassette encoding the lipid-regulating transcription factor. Inactivating mutants of the lipid-regulating transcription factors, created by mutational approaches including, but not limited to, transposon insertion or CRISPR/Cas induced mutagenesis, can reduce lipid content of plants seeds by approximately 7.2% to approximately 18% compared to plant seeds were not mutagenized, and therefore have functional lipid-regulating transcription factor(s).

The lipid-regulating transcription factors can be overexpressed in a variety of plants including plants that are agronomically important sources of oils. One group of closely related species that belong to the Brassicaceae family has high seed lipid content. For example, rape seed, canola, several mustard species, and Camelina sativa are all Brassica species that are commercially grown as oil crops. The model plant species Arabidopsis thaliana is also a member of the Brassicaceae family and therefore serves as a good model to study lipid biosynthesis as well as to explore strategies to increase seed lipid content. As a result, much of the biochemical and genetic basis (i.e., enzymes, pathways, etc.) of lipid biosynthesis was discovered in Arabidopsis over the past three decades.

Transcription factors (TFs) are a special class of genes that is responsible for controlling the level of expression of genes. Most plant species, including Arabidopsis, include approximately 1,500-2,000 TFs in their genome. Each TF is posited to regulate hundreds of genes that are part of multiple pathways. Therefore, TFs serve as control switches to modulate the synthesis of all plant metabolic products and thus the allocation of resources to starch vs. lipid content. Genetic engineering strategies have been used to improve the lipid content of seeds using the over-expression of a few TFs, notably LEC1, FUS3, ABI3, WRI1, MYB96, GmDOF4, GmDOF11, AGL15 and SPT. Many of these TF over-expression strategies have been shown to work across species, e.g., over-expressing the soybean TFs GmDOF4 and GmDOF11 increases seed lipid content in Arabidopsis. Thus, TF over-expression is a viable strategy to improve seed lipid accumulation and works well across plant species.

A computational strategy was developed and deployed to identify TFs that control the expression level of genes involved in lipid biosynthesis in the seeds. This strategy predicts de novo the ability of known TFs to control lipid biosynthesis and hence final lipid content of a seed. This strategy then allows the ranking of all TFs in the order of influence over the synthesis of lipids in the seeds.

The top twenty most influential TFs (Table I) identified by this approach included eight TFs that have been previously shown to alter significantly seed lipid content by mutant and/or over-expression screens. Another eight unknown TFs were identified as good candidates to alter seed lipid content either positively or negatively. TF genes in Table I that are unshaded are previously unknown lipid-regulating TFs. TF genes in Table I that are shaded are known lipid-regulating TFs.

TABLE I

| GeneID | Gene Name | Synonyms |
|---|---|---|
| AT5G08520 | MYBS2 | Duplicated homeodomain-like superfamily protein |
| AT5G65640 | bHLH093 | bHLH093, beta HLH protein 93 |
| AT5G65410 | ATHB25 | ATHB25, HB25, homeobox protein 25, ZFHD2, |
| | | ZINC FINGER HOMEODOMAIN 2, ZHD1, ZINC |
| | | FINGER HOMEODOMAIN 1 |
| AT5G04760 | DiV2 | Duplicated homeodomain-like superfamily protein |
| AT1G25330 | CESTA | CES, CESTA, HAF, HALF FILLED |
| AT5G10030 | TGA4 | OBF4, OCS ELEMENT BINDING FACTOR 4, |
| | | TGA4, TGACG motif-binding factor 4 |
| AT3G60030 | SPL12 | SPL12, squamosa promoter-binding protein-like 12 |
| AT3G57390 | AGL18 | AGL18, AGAMOUS-like 18 |
| AT1G21970 | AtLEC1 | AtLEC1, EMB 212, LEC1, LEAFY COTYLEDON 1, |
| | | NF-YB9, NUCLEAR FACTOR Y, SUBUNIT B9 |
| AT3G27785 | ATMYB118 | ATMYB118, MYB118, myb domain protein 118, |
| | | PGA37, PLANT GROWTH ACTIVATOR 37 |
| AT5G35550 | TT2 | ATMYB123, MYB DOMAIN PROTEIN 123, |
| | | ATTT2, MYB123, MYB DOMAIN PROTEIN 123, |
| | | TT2, TRANSPARENT TESTA 2 |
| AT5G40360 | AtMYB115 | AtMYB115, myb domain protein 115, MYB115, myb |
| | | domain protein 115 |
| AT3G26790 | FUS3 | FUS3, FUSCA 3 |
| AT3G54320 | WRI1 | ASML1, ACTIVATOR OF SPO(MIN)::LUC1, |
| | | ATWRI1, WRI, WRINKLED, WRI1, WRINKLED 1 |
| AT5G62470 | MYB96 | ATMYB96, MYB DOMAIN PROTEIN 96, MYB96, |
| | | myb domain protein 96, MYBCOV1, |
| AT1G16060 | WRI3 | ADAP, ARIA-interacting double AP2 domain protein, |
| | | WRI3, WRINKLED 3 |

| An amino acid sequence of MYBS2 protein from Arabidopsis thaliana is shown | |
|---|---|
| below as SEQ ID NO. 1: | |
| [SEQ ID NO: 1] | |
| MTVEEVSDGS VWSREDDIAF ERALANNTDE SEERWEKIAA | |
| DVPGKSVEQI KEHYELLVED VTRIESGCVP LPAYGSPEGS | |
| NGHAGDEGAS SKKGGNSHAG ESNQAGKSKS DQERRKGIAW | |
| TEDEHRLFLL GLDKYGKGDW RSISRNFVVT RTPTQVASHA | |
| QKYFIRLNSM NKDRRRSSIH DITSVGNADV STPQGPITGQ | |
| NNSNNNNNNN NNNSSPAVAG GGNKSAKQAV SQAPPGPPMY | |
| GTPAIGQPAV GIPVNLPAPP HMAYGVHAAP VPGSVVPGAA | |
| MNIGQMPYTM PRIPTAHR | |
| A nucleotide sequence that encodes the MYBS2 protein with amino acid | |
| sequence SEQ ID NO. 1 is shown as SEQ ID NO. 2: | |
| [SEQ ID NO: 2] | |
| 1 | atgaggagta gcagtaataa aatacatatg gcataatttg gtcaacaagg aaattccaat |
| 61 | atgaattgga agtgtaacaa ataataaaag agtcaacaat accaaaaata acaacagcgg |
| 121 | acttgagctg tgaaaactgt tgcttatggt ttttattcac tgtttctttg ttttgaactt |
| 181 | ccccttcctt taacaatggc gttttgaccc ctccctatct ctctctctct ctctctcagt |
| 241 | cttctgggtt tttcctattc ctctttctct ctctctcctt caagttgctg caatcccttg |
| 301 | aaaacccaat aaacccccaa ttttccattt ctcataaagt tcacattttt ccttcttctt |
| 361 | cttctgccaa ttctctgatt ccctcgtttc aatctccgtt ttgctttgcc tatcagataa |
| 421 | atttcttctt gctttccttt ctatctcata acgataagtt gaattaatct ttgcgtctta |
| 481 | gttcatcagt agagagagta gggttttttc cgcttatttt tagggaattt cattttgttg |
| 541 | gaggtgagaa tctctattcg agtccccaag attctcttta tatccctagt ttagttgtat |
| 601 | ggttgggttt tgattggaat atcaaggggt agtttttagc taggttcact gatacttgga |
| 661 | agatctgagt tctttgggat tctgttaagt ttgtggagat ctaaaagaca cgaaatttgt |
| 721 | agaaatctgg ttgatatccc agacttttag agggattagg gtagattcta tagaatttga |
| 781 | ggcgggtttg attggaatta tgacggtgga ggaagttagt gatggttctg tgtggagtag |
| 841 | ggaggatgat attgcctttg agagagctct agccaataat accgatgaat cagaggaacg |
| 901 | gtgggagaag attgctgcag acgttccagg caaaagtgtt gaacagatta aagaacatta |
| 961 | cgagctttta gttgaagatg ttactaggat tgaatcagga tgtgtgcctc ttcctgccta |
| 1021 | tgggtctcct gaaggatcga atggccatgc tggtgatgaa ggagcaagta gtaagaaagg |
| 1081 | aggtaacagt catgcgggag agtctaacca agcaggtaaa tcaaagtccg atcaagaacg |
| 1141 | acgaaagggt atcgcgtgga cagaagatga gcacaggtta tttcttcttg gtttggataa |
| 1201 | gtacgggaaa ggtgattggc gtagcatttc tcgcaacttt gtagtaacaa gaacaccgac |
| 1261 | ccaagttgcg agccatgctc aaaagtattt cattcgtcta aattcaatga acaaagacag |
| 1321 | aaggcgatca agcattcacg acatcactag tgttggcaac gcagatgtct caacgccaca |
| 1381 | aggaccaatc actggtcaga acaacagcaa taacaacaac aacaacaaca acaacaacag |
| 1441 | ttctcctgct gttgctggag gaggaaacaa atcagccaag caagccgtct ctcaagcacc |
| 1501 | acctggacct cctatgtatg gaacacccgc cataggtcag ccagcagttg gaacaccagt |
| 1561 | gaacctccca gctccacctc acatggctta tggagttcat gcggctccag tccctggctc |
| 1621 | agtggttcct ggtgcagcaa tgaacattgg tcaaatgccg tacaccatgc cgcgtacacc |
| 1681 | aacggctcat aggtaactcg aaagcacctt tgctgtcata gtgcactttg tttttaggtg |
| 1741 | taagaaagaa gatgtgtaaa ggatttagtg aatattcaag cttgttcctt gagtgagttt |
| 1801 | tttttattac ttagtttgtg gggattttgt atgaggtccg aataagatat gaagatgaca |
| 1861 | tgattagttt ccagactcga gaagcaaaaa tactcctgtt tgtatgtgaa cacaataaag |
| 1921 | cctctgttat gagacttaca acaaagcaac attgtatatc ttgttctcac attcaacaat |
| 1981 | ctctttgaat tatcaactgc aacgtgcaat tccttatttt ga |
| An amino acid sequence of bHLH093 protein from Arabidopsis thaliana is shown | |
| below as SEQ ID NO. 3: | |
| [SEQ ID NO: 3] | |
| MELSTQMNVFEELLVPTKQETTDNNINNLSENGGFDHHHHQFFPNGYNIDYLCENNEEED | |
| ENTLLYPSSFMDLISQPPPLLLHQPPPLQPLSPPLSSSATAGATEDYPFLEALQEIIDSS | |
| SSSPPLILQNGQEENFNNPMSYPSPLMESDQSKSFSVGYCGGETNKKKSKKLEGQPSKNL | |
| MAERRRRKRLNDRLSMLRSIVPKISKMDRISILGDAIDYMKELLDKINKLQDEEQELGNS | |
| NNSHHSKLFGDLKDLNANEPLVRNSPKFEIDRRDEDTRVDICCSPKPGLLLSTVNTLETL | |
| GLEIEQCVISCFSDESLQASCSEGAEQRDFITSEDIKQALFRNAGYGGSCL | |
| A nucleotide sequence that encodes the bHLH093 protein with the amino acid | |
| sequence SEQ. ID. NO. 3 is shown as SEQ. ID. NO. 4: | |
| [SEQ ID NO: 4] | |
| 1 | atggaactgt cgactcaaat gaatgtgttc gaagagcttc ttgttccgac aaagcaagaa |
| 61 | acaaccgaca acaacatcaa caatctgagc tttaatggcg gatttgatca tcatcatcat |
| 121 | caattcttcc caaatggata taatattgat tacctctgtt tcaacaatga agaagaggac |
| 181 | gaaaataccc ttttgtatcc ttcttctttc atggatctaa tctctcaacc tcctccattg |
| 241 | cttcttcacc aaccgccacc gttacaacca ctgtcgccgc cgttatcctc ctccgcgacc |
| 301 | gccggagcaa catttgacta cccttttctt gaggctttgc aagagataat tgactcttct |
| 361 | tcctcatcgc ctccattgat ccttcaaaat ggtcaagaag agaactttaa taatccgatg |
| 421 | tcgtatccct ctccattgat ggagtctgat cagagcaaga gcttcagtgt tggttactgt |
| 481 | ggaggagaga cgaacaagaa gaagagcaaa aagcttgaag gccaaccttc taagaatctc |
| 541 | atggcggaga gacgacggag aaaacgactt aacgatcgtc tttctatgct ccgatccatc |
| 601 | gtcccaaaaa tcagtaagat ggacaggaca tcgatattag gagatgccat agattacatg |
| 661 | aaagagcttt tagacaaaat caacaaatta caagatgagg aacaagaact tggaaatagc |
| 721 | aacaattcac atcactctaa gctcttcggt gatctcaagg atcttaatgc gaacgaacct |
| 781 | ctggtcagaa actcaccaaa gtttgaaata gatcgtagag acgaggatac tcgagttgat |
| 841 | atatgctgct cgccaaaacc gggattgcta ctatctactg tgaatacatt agagactcta |
| 901 | ggcttggaga ttgaacaatg tgttataagc tgctttagtg atttctcttt gcaggcttct |
| 961 | tgttctgagg gagctgagca gagagatttc ataacatcag aagatataaa acaagcatta |
| 1021 | ttcagaaacg caggttatgg tggaagctgc ttgtaa |
| An amino acid sequence of Arabidopsis thaliana (AT) HB25 protein is shown | |
| below as SEQ ID NO. 5: | |
| [SEQ ID NO: 5] | |
| MEFEDNNNNNDEEQEEDMNLHEEEEDDDAVYDSPPLSRVLPKASTESHETTGTTSTGGGGGFMV | |
| VHGGGGSRFRFRECLKNQAVNIGGHAVDGCGEFMPAGIEGTIDALKCAACGCHRNFHRKELPYF | |
| HHAPPQHQPPPPPPGFYRLPAPVSYRPPPSQAPPLQLALPPPQRERSEDPMETSSAEAGGGIRK | |
| RHRTKFTAEQKERMLALAERIGWRIQRQDDEVIQRFCQETGVPRQVLKVWLHNNKHTLGKSPSP | |
| LHHHQAPPPPPPQSSFHHEQDQP | |
| A nucleotide sequence that encodes the ATHB25 protein with the amino acid | |
| sequence SEQ ID NO. 5 is shown as SEQ ID NO. 6: | |
| [SEQ ID NO: 6] | |
| 1 | acattatctc tctcttcttc ttcaccttct tgagaaacct cctctcccca aaaacgacgt |
| 61 | agtttcacat ttctcgactt cttgaatgga gtttgaagac aacaacaaca acaacgacga |
| 121 | agagcaagaa gaggatatga atcttcatga ggaagaagaa gacgacgacg ccgtttacga |
| 181 | ctctcctcct ctctctcgtg ttctccccaa agcctcgaca gaaagtcatg aaaccaccgg |
| 241 | aactacttcc acaggcggtg gcggaggatt catggttgtt cacggcggtg gagggagcag |
| 301 | gtttaggttc cgtgagtgtc tcaagaacca agcggtgaac ataggaggac acgcggtcga |
| 361 | tggttgtggt gagtttatgc cagctggaat cgaaggtacc atcgacgctc taaaatgcgc |
| 421 | cgcttgtggc tgtcaccgta acttccaccg caaggaatta ccttacttcc atcacgcgcc |
| 481 | gccacaacat cagcctcctc ctcccccgcc agggttttac cgtcttccag ctccggttag |
| 541 | ctaccgacca ccaccgtcac aagctcctcc tcttcagctc gctcttcccc ctccacaaag |
| 601 | agagagatca gaagatccaa tggagacgtc ttcagctgaa gcaggaggag ggattaggaa |
| 661 | gaggcatagg actaagttta cggctgagca aaaggaaagg atgttagctt tagctgagag |
| 721 | gattggatgg agaattcaga gacaagacga tgaagtgatt cagagatttt gtcaggagac |
| 781 | tggtgttccg agacaagttc ttaaggtttg gttacataac aacaaacaca ctcttggtaa |
| 841 | gtcgccttca ccacttcatc atcatcaggc tcctcctcct ccaccaccac agtcttcgtt |
| 901 | tcatcatgaa caagaccaac catgaatctt gaatttcttt gatcactagg gttttaattt |
| 961 | agcttaatta attacttgag aaatttgaga gacaaggttt ttattgttta atttatgtac |
| 1021 | ccattttcct ctttgatgat gatgttgatg atgttggtga tgatctttaa tttctggtta |
| 1081 | attatgtttt |
| An amino acid sequence of DiV2 protein from Arabidopsis thaliana is shown | |
| below as SEQ ID NO. 7: | |
| [SEQ ID NO: 7] | |
| MASSQWTRSEDKMFEQALVLFPEGSPNRWERIADQLHKSAGEVREHYEVLVHDVFEIDSGR | |
| VDVPDYMDDSAAAAAGWDSAGQISFGSKHGESERKRGTPWTENEHKLFLIGLKRYGKGDWR | |
| SISRNVVVTRIPTQVASHAQKYFLRONSVKKERKRSSIHDITTVDATLAMPGSNMDWTGQH | |
| GSPVQAPQQQQIMSEFGQQLNPGHFEDFGFRM | |
| A nucleotide sequence that encodes the DiV2 protein with the amino acid | |
| sequence SEQ ID NO. 7 is shown as SEQ ID NO. 8: | |
| [SEQ ID NO: 8] | |
| 1 | aactgagaga aagagagaga caaaagacat cccaattgca gagaaaccgt gttgaagttg |
| 61 | gtctctgagc tgcttcttcc ttcccttttt tatttttatt tttccagctc tttattttct |
| 121 | tatttacaaa aaactttccc aagaataaaa accagaagaa tccgtataaa ttatcctaac |
| 181 | agtttcttcc aatatccaac aaatttcaga tttttgtttt tgttttcttc ttctctactt |
| 241 | gagtaatcat caacgattat ggcgtcaagt cagtggacga ggtcggagga taagatgttt |
| 301 | gagcaagctt tggttctttt tcctgaagga tctcctaatc ggtgggagag aatcgctgat |
| 361 | cagcttcata aatctgctgg tgaagttagg gagcattacg aggtcttggt tcatgatgtt |
| 421 | ttcgagattg attctggtcg agttgatgtc cctgattaca tggatgactc ggcggctgcg |
| 481 | gcggcgggtt gggattccgc tggtcagatc tcttttgggt ctaaacatgg cgagagtgaa |
| 541 | cgcaaaagag gaactccttg gacagagaac gaacacaaat tgtttctgat cggattaaag |
| 601 | agatatggta agggagattg gaggagtatc tcgagaaacg ttgtggtgac gaggacaccg |
| 661 | acgcaagtcg cgagtcacgc tcagaagtat tttctgagac agaactcggt gaagaaggag |
| 721 | aggaaaaggt cgagcatcca tgatataact acggttgatg ctactttggc tatgcctggg |
| 781 | tctaacatgg actggactgg ccaacacggg agtcctgttc aggcgccgca gcagcaacag |
| 841 | attatgtctg agttcggtca gcaattgaat cctggtcatt tcgaggattt tgggtttcgg |
| 901 | atgtgatgaa gaaaggggga gacaaattgg gatggcttta gtttaatagg gttttactca |
| 961 | ttttatgtga atagggaaag aaataggatt gggtattttc ttatacagat gatgatgatg |
| 1021 | atgatcaaag aataaaaata atgtataggg aagctttttg taaacacaaa tatgtttggt |
| 1081 | aaccattttg gtattttgaa tcaatgcata tggttgtttt tctcgatt |
| An amino acid sequence of CESTA protein from Arabidopsis thaliana is | |
| shown below as SEQ ID NO. 9: | |
| [SEQ ID NO: 9] | |
| MARFEPYNYNNGHDPFFAHINQNPELINLDLPASTPSSFMLFSNGALVDANHNNSHFFPNLL | |
| HGNTRRKGNKEESGSKRRRKRSEEEEAMNGDETQKPKDVVHVRAKRGQATDSHSLAERVRRE | |
| KINERLKCLQDLVPGCYKAMGMAVMLDVIIDYVRSLQNQIEFLSMKLSAASACYDLNSLDIE | |
| PTDIFQGGNIHSAAEMERILRESVGTQPPNFSSTLPF | |
| A nucleotide sequence that encodes the CESTA protein with the amino acid | |
| sequence SEQ ID NO. 9 is shown as SEQ ID NO. 10: | |
| [SEQ ID NO: 10] | |
| 1 | ataactgtct tagagaaaga aaaaaaacaa aactagctca caaaaaggaa atcatatttt |
| 61 | gatttaattt gtagtgtctc taatggcacg gtttgagcca tataactata ataatggtca |
| 121 | tgatcctttc tttgcacaca ttaaccaaaa tccagagcta ataaatctgg acttaccagc |
| 181 | ttctacccct tccagtttca tgcttttctc caatggagct ttagttgatg ccaatcacaa |
| 241 | taattctcac ttcttcccaa atttattgca cggtaatacg agaagaaaag gaaataaaga |
| 301 | agagagtggg tcgaagagaa gaagaaagag gtcggaagag gaagaagcca tgaatggaga |
| 361 | tgagactcag aagccaaaag atgttgttca tgtccgagct aagagaggtc aagctactga |
| 421 | tagccatagt ttggctgaaa gggtacgaag agagaagatc aatgaaaggc tgaaatgctt |
| 481 | acaagacctt gttccaggat gctacaaggc aatgggaatg gcagtgatgc ttgatgtcat |
| 541 | catagattat gtacgatcac tccagaatca aatcgagttt ttgtccatga aactctcagc |
| 601 | ggcaagtgca tgttacgacc ttaattcttt ggatattgag ccaacggata tatttcaggg |
| 661 | agggaatatt catagtgcag cagagatgga aaggatttta agagaaagcg ttggaacaca |
| 721 | gcctcctaat ttcagttcaa cattaccctt ttgatcataa gaaaattatg aattttcaga |
| 781 | gaaattattc tctttttcta taattaaact ccataaataa ggacttacca tgatcagtat |
| 841 | atataggttt atctatcttt tgtgtgtgac gtcagtatac ttttatcata aatgtgtaac |
| 901 | cttatgatta tgaagcttat ccatatagta tgtaccatga aaatgagtaa agctatatgt |
| 961 | tacaaagaaa ctctatttga agtaacataa gatttcgata tt |
| An amino acid sequence of TGA4 protein from Arabidopsis thaliana is shown | |
| below as SEQ ID NO. 11: | |
| [SEQ ID NO: 11] | |
| MNTISTHFVPPRRFEVYEPLNQIGMWEESFKNNGDMYTPGSIIIPTNEKPDSLSEDISHGTE | |
| GTPHKFDQEASTSRHPDKIQRRLAQNREAARKSRLRKKAYVQQLETSRLKLIHLEQELDRAR | |
| QQGFYVGNGVDINALSFSDNMSSGIVAFEMEYGHWVEEQNRQICELRTVLHGQVSDIELRSL | |
| VENAMKHYFQLFRMKSAAAKIDVFYVMSGMWKISAERFFLWIGGFRPSELLKVLLPHFDPLT | |
| DQQLLDVCNLRQSCQQAEDALSQGMEKLQHTLAESVAAGKLGEGSYIPQMTCAMERLEALVS | |
| FVNQADHLRHETLQQMHRILITRQAARGLLALGEYFQRLRALSSSWAARQREPT | |
| A nucleotide sequence that encodes the TGA4 protein with the amino acid | |
| sequence SEQ ID NO. 11 is shown as SEQ ID NO. 12: | |
| [SEQ ID NO: 12] | |
| 1 | gagaaataca gaataaccag aaacaaaatt aaattagtaa tcgaataaat tatggactta |
| 61 | aaggtatctt aaacatgtca aaatttggtt tttgactgtg tagctgatgt atctagtaca |
| 121 | tatacttaaa ggacaaatat caccgaagaa tcaacaaacc aaaaaaaaaa acagaaataa |
| 181 | atgttagtat atatattttt attgacattt attaggatat agaaaaattt agaaaactca |
| 241 | atcaaaggtc tctcttttaa agttgtcgtg ttctctcttg aatgattctt cttctccttc |
| 301 | ttcgagatga caagttcaga gaacgagatt ttaccatccc ttattctatc agaccggttt |
| 361 | aagctgcaga tttctaggct tagcgttcga tttcgtcgct gaaagtgaaa agttcatcta |
| 421 | gctttagttt tctcttttca tggtttccgc gggaaaagtt cgtctttttt gaagcccttt |
| 481 | tgacacaaaa gaccagaaca agttgaagaa atatgaatac aacctcgaca cattttgttc |
| 541 | caccgagaag gtttgaagtt tacgagcctc tcaaccaaat cggtatgtgg gaagaaagtt |
| 601 | tcaagaacaa tggagacatg tatacgcctg gctctatcat aatcccgact aacgaaaaac |
| 661 | cagacagctt gtcagaggat acttctcatg ggacagaagg aactcctcac aagtttgacc |
| 721 | aagaggcttc cacatctaga catcctgata agatacagag aaggctagca cagaatcgag |
| 781 | aggcagctag gaaaagtcgt ttgcgcaaga aagcttatgt tcagcagcta gagactagcc |
| 841 | ggttaaagct aattcattta gagcaagaac tcgatcgtgc tagacaacag ggtttctatg |
| 901 | tggggaacgg agtagatacc aatgctctta gtttctcaga taacatgagc tcagggattg |
| 961 | ttgcatttga gatggaatat ggacattggg tggaagaaca gaacaggcaa atatgtgaac |
| 1021 | taagaacggt tttacatgga caagttagtg atatagagct tcgttctcta gtcgagaatg |
| 1081 | ccatgaaaca ttactttcaa ctcttccgaa tgaagtcagc cgctgcaaaa atcgatgttt |
| 1141 | tctatgtcat gtccggaatg tggaaaactt cagcagagcg gtttttcttg tggataggcg |
| 1201 | gatttagacc ctcagagctt ctcaaggttc tgttaccgca ttttgatcct ttgacggatc |
| 1261 | aacaactttt ggatgtatgt aatctgaggc aatcatgtca acaagcagaa gatgcgttat |
| 1321 | cccaaggtat ggagaaactg caacatacat tagcagagag tgtagcagcc gggaaacttg |
| 1381 | gtgaaggaag ttatattcct caaatgactt gtgctatgga gagattggag gctttggtca |
| 1441 | gctttgtaaa tcaagctgat catctgagac atgagacatt gcaacagatg catcggatct |
| 1501 | taaccacgcg acaagcggct agaggtttgt tagcattagg ggagtatttc caaaggcttc |
| 1561 | gagctttgag ttcgagttgg gcggctaggc aacgtgaacc aacgtaatta aggtgtttag |
| 1621 | atgtcaagaa aggtttgaga ccttaacaat caagaatgga gtttgctggt gagtggattt |
| 1681 | ttgggtcaag aacaagagca ataacacaag ctgctgtgtg atgatgaatc ttgtcttgcg |
| 1741 | gctaaaggaa atgtttgagg aaagttgtac atatgatcag caacgtaaag tttatagctt |
| 1801 | tttagaaacc aacttttcga tggttgttct tttttttttg tatgtaatat tatagataag |
| 1861 | cttgtggtat atatgatttt aatgtgacat tacgaacttg atttataacc atggtaaaat |
| 1921 | tatttaacaa aatatgtaat aatagaatgg gggaagggga ataaat |
| An amino acid sequence of SPL12 protein from Arabidopsis thaliana is shown | |
| below as SEQ ID NO. 13: | |
| [SEQ ID NO: 13] | |
| MEARIEGEVEGHSLEYGFSGKRSVEWDLNDWKWNGDLFVATQLN | |
| HGSSNSSSICSDEGNVEIMERRRIEMEKKKKRRAVTVVAMEEDNLKDDDAHRLILNLG | |
| GNNIEGNGVKKIKLGGGIPSRAICCQVDNCGADLSKVKDYHRRHKVCEIHSKATTALV | |
| GGIMQRFCQQCSRFHVLEEFDEGKRSCRRRLAGHNKRRRKANPDTIGNGISMSDDQTS | |
| NYMLITLLKILSNIHSNQSDQTGDQDLLSHLLKSLVSQAGEHIGRNLVGLLQGGGGLQ | |
| ASQNIGNLSALLSLEQAPREDIKHHSVSETPWQEVYANSAQERVAPDRSEKQVKVNDF | |
| DINDIYIDSDDTTDIERSSPPPINPATSSLDYHQDSRQSSPPQTSRRNSDSASDQSPS | |
| SSSGDAQSRIDRIVFKLFGKEPNDFPVALRGQILNWLAHTPTDMESYIRPGCIVLTIY | |
| LRQDEASWEELCCDLSFSLRRLLDLSDDPLWTDGWLYLRVQNQLAFAFNGQVVLDTSL | |
| PLRSHDYSQIITVRPLAVTKKAQFTVKGINLRRPGTRLLCTVEGTHLVQEATQGGMEE | |
| RDDLKENNEIDFVNFSCEMPIASGRGFMEIEDQGGLSSSFFPFIVSEDEDICSEIRRL | |
| ESTLEFTGIDSAMQAMDFIHEIGWLLHRSELKSRLAASDHNPEDLFSLIRFKFLIEFS | |
| MDREWCCVMKKLLNILFEEGTVDPSPDAALSELCLLHRAVRKNSKPMVEMLLRFSPKK | |
| KNQTLAGLFRPDAAGPGGLTPLHIAAGKDGSEDVLDALTEDPGMTGIQAWKNSRDNTG | |
| FTPEDYARLRGHFSYIHLVQRKLSRKPIAKEHVVVNIPESFNIEHKQEKRSPMDSSSL | |
| EITQINQCKLCDHKRVFVTTHHKSVAYRPAMLSMVAIAAVCVCVALLFKSCPEVLYVF | |
| QPFRWELLEYGTS | |
| A nucleotide sequence that encodes the SPL12 protein with the amino acid | |
| sequence SEQ ID NO. 13 is shown as SEQ ID NO. 14: | |
| [SEQ ID NO: 14] | |
| 1 | attttttctt atttaaaaag gcgaaatcga gtgagacacg aaatatgggg aagtggtccc |
| 61 | caccattatt tccatttttt tatttcttct tcttcttctt ctctgtttct ctttctgtgt |
| 121 | cgaaatgaag atgctttaaa tctaacttct tttttttgtt tgggcactat aaaaccatat |
| 181 | ccttaccgct accaaacgct tctcctttaa tcatcatcat catcatttta tctccgtccc |
| 241 | ttcatgttcc gttaccgtta acggctGttt ttttttaatc tccggtgacc gttctttttc |
| 301 | tgattccagg tgttgccagc tgtttttttt tttgggagat atttttttta attcctttta |
| 361 | aagttagtta taagtcagtt ttgtctgctt ggattttcat ttataaaaca aggtggaatt |
| 421 | aggtttcttc cgatagagaa aggggcttga ttttggacaa aggcacgtgt cttagtcttt |
| 481 | tattcaagct ccgatctata agttatgatt tgggtttggt tttgagttct ccaattttag |
| 541 | tctcatctct gtggttgttg taactgggtt tgtgatggaa gctagaattg aaggtgaagt |
| 601 | agaaggtcac agtttagaat acgggtttag tggtaaaagg agtgtagagt gggatttgaa |
| 661 | tgattggaaa tggaatggtg atctcttcgt tgctacacag ctgaatcacg gctcatccaa |
| 721 | cagctcttct acgtgctctg atgaaggaaa tgttgaaatc atggagagaa ggaggataga |
| 781 | gatggagaag aagaagaaaa gaagagctgt tactgtggta gcaatggaag aagataactt |
| 841 | gaaagatgat gatgctcata gacttactct gaatcttggt ggtaataata ttgaggggaa |
| 901 | tggtgtgaaa aagacgaaac ttggaggagg gattccgagt cgggcgattt gttgtcaggt |
| 961 | agacaactgt ggagctgatt taagcaaagt taaggattat catagacgtc ataaggtctg |
| 1021 | tgagattcat tctaaagcta ctactgcact tgttggagga attatgcagc ggttttgtca |
| 1081 | gcaatgtagt aggtttcatg tgcttgaaga gtttgatgag ggaaagagaa gttgccgtag |
| 1141 | acgtttggct gggcataata agcgtagaag aaaagcaaat cctgatacta taggcaatgg |
| 1201 | gacttctatg agtgatgatc aaacaagcaa ttatatgttg attactctct tgaagatact |
| 1261 | ctccaatatt cattcgaatc agtcggatca aacgggtgat caggatctac tgtctcatct |
| 1321 | tctaaagagc cttgtaagcc aagctggtga acatataggg aggaatttag ttgggcttct |
| 1381 | acagggtgga ggaggactcc aggcttctca aaatattgga aacttatccg ctttgctctc |
| 1441 | actcgagcaa gcccctcgag aggatataaa acatcattca gtgtctgaaa cgccttggca |
| 1501 | agaagtgtat gccaatagtg ctcaagagag agttgccccg gataggtccg agaaacaagt |
| 1561 | caaagtgaat gattttgatt tgaatgacat ctacatagac tcagatgata ccacagatat |
| 1621 | agagagatca tcacctcctc cgacaaatcc agctactagt tctcttgatt atcatcaaga |
| 1681 | ctcgcgtcag tctagtccac ctcaaactag taggaggaat tcagattcag cttctgacca |
| 1741 | atcaccttca agttccagtg gagatgcaca gagccgtact gatcggattg tgttcaaact |
| 1801 | ctttgggaaa gagccgaatg attttccagt tgccttacga ggacagattc ttaactggtt |
| 1861 | agcgcatact ccaactgata tggagagcta cattagacct ggttgtatcg ttttgaccat |
| 1921 | ttatcttcgt caagatgaag cttcttggga agaactttgt tgtgatctga gtttcagctt |
| 1981 | gaggaggctt ctagatcttt cagatgatcc tttatggact gatgggtggc tttatcttag |
| 2041 | ggtgcaaaac caactcgcat ttgcgtttaa tggtcaggtt gttcttgaca catctttacc |
| 2101 | tctgagaagc catgattata gccaaatcat tactgttaga ccacttgctg taacaaagaa |
| 2161 | agctcaattt acagtaaaag gcatcaatct ccgtcgacct ggcacaaggt tactttgtac |
| 2221 | tgttgaagga acacacttgg ttcaggaagc aacacaagga gggatggagg agagagatga |
| 2281 | tctaaaggag aataatgaga ttgattttgt caatttttcc tgtgagatgc ctattgcaag |
| 2341 | tggtcgaggt ttcatggaaa ttgaagacca aggtgggcta agcagtagtt tcttcccttt |
| 2401 | catagtatct gaagacgaag atatttgttc tgaaatccga agactcgaaa gcacattaga |
| 2461 | gtttactgga actgattctg caatgcaagc aatggatttc atccacgaaa tcggttggct |
| 2521 | tctacacaga agtgaactca agtcaagact tgcagcatca gatcataatc cagaggatct |
| 2581 | gttttcttta atacggttta agtttctaat agagttctca atggaccggg aatggtgctg |
| 2641 | tgtgatgaag aagttattga atattctatt cgaagaaggc actgttgatc catctcctga |
| 2701 | tgctgcgtta tcagaactgt gtcttcttca cagagccgtg aggaaaaatt caaaacccat |
| 2761 | ggttgagatg cttcttagat tcagtcccaa gaagaagaat cagacattag ccggtttgtt |
| 2821 | tagacccgat gcagctggtc caggcggttt aacaccgctt cacattgcag ctggtaaaga |
| 2881 | cggttcagaa gatgtgttgg atgccctgac tgaggatcct ggaatgactg gtattcaagc |
| 2941 | ctggaaaaat tcccgggaca ataccggatt cacaccagaa gactatgcac gtttacgagg |
| 3001 | tcacttctcg tatattcatc tggtgcaacg gaaactcagt cggaaaccaa tcgccaaaga |
| 3061 | acacgtggtg gtcaacattc ctgagtcttt caacattgag cacaaacaag aaaagaggag |
| 3121 | tccaatggat tcttcaagct tggagataac acagataaac caatgtaagc tctgtgatca |
| 3181 | caaacgtgtg tttgtcacaa cgcaccacaa gtctgttgcc tacaggccag caatgctttc |
| 3241 | gatggtagcg atagcagcgg tttgcgtttg tgtcgctctt ttgttcaaga gttgcccgga |
| 3301 | agtgctatat gtctttcagc cattcaggtg ggaattactt gagtatggaa caagctagta |
| 3361 | aaacttccct ataccagaca ttgaaaaaga atcttctcaa tagaagatgt gttggtgtga |
| 3421 | aatctacgag tgtaatttta tgtaacgttt aattattttt actagaatat gtatcattca |
| 3481 | gctttacaag attatatgta atgtagcttt atctgatgtt aaacccccga atttgtataa |
| 3541 | ctacgttttt gtgtgtttgt tacattttgt actttgtctt tgatggcaaa tcttgaagtg |
| 3601 | agtgagaata acaacaaatc acataaaaca ccaa |
| An amino acid sequence of AGL18 protein from Arabidopsis thaliana is shown | |
| below as SEQ ID NO: 15: | |
| [SEQ ID NO: 15] | |
| MGRGRIEIKKIENINSRQVTFSKRRNGLIKKAKELSILCDAEVALIIFSSIGKIYDFSSVCME | |
| QILSRYGYTTASTEHKQQREHQLLICASHGNEAVLRNDDSMKGELERLQLAIERLKGKELEGM | |
| SFPDLISLENQLNESLHSVKDQKTQILLNQIERSRIQEKKALEENQILRKQVEMLGRGSGPKV | |
| LNERPQDSSPEADPESSSSEEDENDNEEHHSDTSLQLGLSSTGYCTKRKKPKIELVCDNSGSQ | |
| VASD | |
| A nucleotide sequence that encodes the AGL18 protein with the amino acid | |
| sequence SEQ ID NO. 15 is shown as SEQ ID NO. 16: | |
| [SEQ ID NO: 16] | |
| 1 | gaaaaaccct agaagataaa aaaaaaggga ggcgatacac gaaatagaga agagatccaa |
| 61 | aaccaaaaca agggcgttat aaatgggaag aaagttccaa aagaagaaaa cagccactca |
| 121 | caccaagcaa agcaaattag caaagccacc aaatatggaa aattgctact tatagtaact |
| 181 | cccatctttt tatataatac cctaattttg gtcctaattt tccttcctta gcccctttcc |
| 241 | caatatttga tcaatttcta aagaaaaccc cttcctctac tagtcctcct cctatatata |
| 301 | caaaatctta agaaatctct ctacttgttt cctctgttat cataatctct tctctctata |
| 361 | tctcttctct tcttctttta ccctgttttt tttttcattc cacagagccc aggttgattg |
| 421 | attttgttat tcagagatat ggggagagga aggattgaga ttaagaagat tgagaatatc |
| 481 | aacagtcgtc aagtcacttt ctctaagaga cgaaacggtt tgatcaagaa ggctaaagag |
| 541 | ctttcgattc tctgtgacgc cgaggttgct cttatcatct tctccagcac cggcaagatt |
| 601 | tacgatttct ccagcgtctg tatggagcaa attctttcta gatatggata cactactgcg |
| 661 | tccactgagc ataaacaaca aagagaacac caacttctaa tttgtgcttc acatggaaat |
| 721 | gaagctgtgt tgcgaaatga tgattctatg aagggggaac ttgaaagatt acagcttgca |
| 781 | attgagagac ttaagggtaa ggagcttgaa ggtatgagtt tcccggatct tatttctctt |
| 841 | gaaaaccagt tgaacgagag cttgcatagt gtcaaggatc aaaagacaca aatcctgctc |
| 901 | aaccagattg agagatccag gatacaggag aaaaaagcat tggaagaaaa ccaaatcttg |
| 961 | cgcaaacagg ttgagatgtt ggggagaggt tcaggaccaa aagtgttgaa tgaaaggcct |
| 1021 | caagattcta gcccagaagc cgatcccgag agctcttcat cagaagagga tgagaatgac |
| 1081 | aacgaggagc accattccga cacttccttg cagttggggt tgtcgtcgac ggggtattgc |
| 1141 | acaaagagaa agaagccgaa gatcgaactg gtctgcgata actctgggag tcaagtggct |
| 1201 | tctgattgat ggaatcgatt atttttctaa ttctggttgt ttaggggtct ctatgtgtct |
| 1261 | tcttgtttct ggctgttctt ttgctttatt tcatctcaag tagagttttc ttaatgttta |
| 1321 | ggtggaacat ttttccataa tcaagaaggg atttgatcaa tcaataacat tagattttct |
| 1381 | tagttaaaga cttaaagttg cccacacacc acaccatatg tgattatgat gaatttacat |
| 1441 | tttataaaca gg |

The nucleic acids and polypeptides allow identification and isolation of related nucleic acids and their encoded proteins that provide for production of healthy plants with modulated lipid content, such as increased lipid content.

Plant cells, plant seeds, and plants disclosed herein can comprise an expression cassette comprising a promoter operably linked to a nucleic acid segment encoding a polypeptide for MYBS2, bHLH093, ATHB25, DiV2, CESTA1, TGA4, SPL12, or AGL18, or a combination thereof, such as any two, three, four, five, six, seven or all eight transcription factors. The polypeptide for MYBS2 has at least about 95% (such as at least 95%, 96%, 97%, 98% or 99%) sequence identity to SEQ ID NO: 1; the polypeptide for bHLH093 has at least about 95% (such as at least 95%, 96%, 97%, 98% or 99%) sequence identity to SEQ ID NO: 3; the polypeptide for ATHB25 has at least about 95% (such as at least 95%, 96%, 97%, 98% or 99%) sequence identity to SEQ ID NO: 5; the polypeptide for DiV2 has at least about 95% (such as at least 95%, 96%, 97%, 98% or 99%) sequence identity to SEQ ID NO: 7; the polypeptide for CESTA1 has at least about 95% (such as at least 95%, 96%, 97%, 98% or 99%) sequence identity to SEQ ID NO: 9; the polypeptide for TGA4 has at least about 95% (such as at least 95%, 96%, 97%, 98% or 99%) sequence identity to SEQ ID NO: 11; the polypeptide for SPL12 has at least about 95% (such as at least 95%, 96%, 97%, 98% or 99%) sequence identity to SEQ ID NO: 13; and the polypeptide for AGL18 has at least about 95% (such as at least 95%, 96%, 97%, 98% or 99%) sequence identity to SEQ ID NO: 15.

Inactivating mutants of MYBS2, bHLH093, ATHB25, DiV2, CESTA1, TGA4, SPL12, and/or AGL18 genes can include gene mutations that interrupt the gene to make a non-functional gene such that expression of the gene is prevented (e.g., gene knockout). For example, inactivating mutations can include gene interruptions including, but not limited to, frameshift mutations, insertion of transposable gene elements, introduction of a stop codon into the gene to stop all downstream transcription (e.g., nonsense mutation), missense mutations, or splice-site mutations. The inactivating mutation can also include loss-of-function mutations that produce a protein having less or no function. For example, loss-of-function mutations can include mutations that result in amino acid changes that interfere with proper protein folding or that interfere with the protein's ability to bind other proteins or to bind DNA.

The MYBS2, bHLH093, ATHB25, DiV2, CESTA1, TGA4, SPL12, and/or AGL18 nucleic acids described herein can include any nucleic acid that can selectively hybridize to a nucleic acid having the nucleotide sequence of any of SEQ ID Nos: 2, 4, 6, 8, 10, 12, 14, and 16 under stringent conditions. Desirably, the nucleic acid that can selectively hybridize to a nucleic acid described herein encodes a polypeptide having activity characteristic of the polypeptide encoded by the nucleic acid to which it hybridizes. The activity can be the same or modulated, such as increased.

The term “selectively hybridize” includes hybridization, under stringent hybridization conditions, of a nucleic acid sequence to a specified nucleic acid target sequence (e.g., any of the SEQ ID NOs: 2, 4, 6, 8, 10, 12, 14, and 16 nucleic acids) to a detectably greater degree (e.g., at least 2-fold over background) than its hybridization to non-target nucleic acid sequences. Such selective hybridization substantially excludes non-target nucleic acids. Selectively hybridizing sequences typically have at least about 60% (such as 60%) sequence identity, at least about 70% (such as 70%) sequence identity, at least about 75% (such as 75%), at least about 80% (such as 80%), at least about 85% (such as 85%), at least about 90% (such as 90%), at least about 95% (such as 95%), at least about 96% (such as 96%), at least about 97% (such as 97%), at least about 98% (such as 98%), at least about 99% (such as 99%), at least about 60% to at least about 99% (such as about 60% to 99%, 60% to about 99%, or 60%-99%) sequence identity, at least about 70% to at least about 99% (such as about 70% to 99%, 70% to about 99%, or 70%-99%) sequence identity, at least about 80% to at least about 99% (such as about 80% to 99%, 80% to about 99%, or 80%-99%) sequence identity, at least about 90% to at least about 99% (such as about 90% to 99%, 90% to about 99%, or 90%-99%), at least about 90% to about 95% (such as about 90% to 95%, 90% to about 95%, or 90%-95%) sequence identity, at least about 95% to about 99% (such as about 95% to 99%, 95% to about 99%, or 95%-99%) sequence identity, at least about 95% to about 97% (such as about 95% to 97%, 95% to about 97% or 95%-97%) sequence identity, at least about 97% to about 99% (such as about 97% to 99%, 97% to about 99% or 97%-99%) sequence identity, or 100% sequence identity (or complementarity) with each other. In some embodiments, a selectively hybridizing sequence has at least about 80% (such as 80%, 85%, 90%, 92.5%, 95%, 97.5%, 98% or 99%) sequence identity or complementarity with SEQ ID NO: 2, 4, 6, 8, 10, 12, 14, or 16.

Thus, the nucleic acids include those with about 500 of the same nucleotides as SEQ ID NO: 2, 4, 6, 8, 10, 12, 14, or 16, or about 600 of the same nucleotides, or about 700 of the same nucleotides, or about 800 of the same nucleotides, or about 900 of the same nucleotides, or about 1000 of the same nucleotides, or about 1100 of the same nucleotides, or about 1200 of the same nucleotides as SEQ ID SEQ ID NO: 2, 4, 6, 8, 10, 12, 14, or 16. The identical nucleotides can be distributed throughout the nucleic acid or the protein, and need not be contiguous.

Note that if a value of a variable that is necessarily an integer, e.g., the number of nucleotides or amino acids in a nucleic acid or protein, respectively, is described as a range, for example 90-99% sequence identity, what is meant is that the value can be any integer between 90 and 99 inclusive, i.e., 90, 91, 92, 93, 94, 95, 96, 97, 98 or 99, or any range between 90 and 99 inclusive, e.g., 91-99%, 91-98%, 92-99%, etc.

### Promoters

The MYBS2, bHLH093, ATHB25, DiV2, CESTA1, TGA4, SPL12, or AGL18 nucleic acids can be operably linked to a promoter, which provides for expression of mRNA from the MYBS2, bHLH093, ATHB25, DiV2, CESTA1, TGA4, SPL12, or AGL18 nucleic acids. The promoter is typically a promoter functional in plants and/or seeds and can be a promoter functional during plant growth and development. A MYBS2, bHLH093, ATHB25, DiV2, CESTA1, TGA4, SPL12, and/or AGL18 nucleic acid is operably linked to the promoter when it is located downstream from the promoter, thereby forming an expression cassette.

Most endogenous genes have regions of DNA that are known as promoters, which regulate gene expression. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences, such as enhancer sequences, that can influence the level of gene expression. Some isolated promoter sequences can provide for gene expression of heterologous DNA, i.e., DNA that differs from the native or homologous DNA.

Promoter sequences are also known to be strong or weak, constitutive or inducible. A strong promoter provides for a high level of gene expression, whereas a weak promoter provides for a low level of gene expression. An inducible promoter is a promoter that allows gene expression to be turned on, for example, in response to an exogenously added agent or to an environmental or developmental stimulus. A bacterial promoter, such as the Ptac promoter, can be induced to vary levels of gene expression depending on the level of isothiopropylgalactoside added to transformed cells. Promoters can also provide for tissue-specific or developmental regulation. An isolated promoter sequence that is a strong promoter for heterologous DNAs is advantageous because it provides for a sufficient level of gene expression for easy detection and selection of transformed cells and provides for a high level of gene expression when desired. A constitutive promoter is a promoter that is unregulated and allows for continual transcription of a gene.

Expression cassettes generally include, but are not limited to, a plant promoter such as the CaMV 35S promoter (Odell et al., Nature. 313:810 812 (1985)), or others such as CaMV 19S (Lawton et al., Plant Molecular Biology. 9:315 324 (1987)), nos (Ebert et al., Proc. Natl. Acad. Sci. USA. 84:5745 5749 (1987)), Adh1 (Walker et al., Proc. Natl. Acad. Sci. USA. 84:6624 6628 (1987)), sucrose synthase (Yang et al., Proc. Natl. Acad. Sci. USA. 87:4144 4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579 589 (1989)) or those associated with the R gene complex (Chandler et al., The Plant Cell. 1:1175 1183 (1989)). Further suitable promoters include the poplar xylem-specific, secondary cell wall-specific cellulose synthase 8 promoter, cauliflower mosaic virus promoter, the Z10 promoter from a gene encoding a 10 kDa zein protein, a Z27 promoter from a gene encoding a 27 kDa zein protein, inducible promoters, such as the light-inducible promoter derived from the pea rbcS gene (Coruzzi et al., EMBO J. 3:1671 (1971)) and the actin promoter from rice (McElroy et al., The Plant Cell. 2:163 171 (1990)). Seed specific promoters, such as the phaseolin promoter from beans, may also be used (Sengupta Gopalan, Proc. Natl. Acad. Sci. USA. 83:3320 3324 (1985)).

### Transformation of Plant Cells

Mutations can be introduced into any one or more of the MYBS2, bHLH093, ATHB25, DiV2, CESTA1, TGA4, SPL12, and AGL18 plant genes by introducing targeting vectors, T-DNA, transposons, nucleic acids encoding TALENS, CRISPR, or ZFN nucleases, and combinations thereof into a recipient plant cell to create a transformed cell. In addition, plant cells can be transformed to include one or more TF transgenes, for example, by transformation of the plant cells with an expression cassette or expression vector.

The frequency of occurrence of cells taking up exogenous (foreign) DNA can sometimes be low. However, certain cells from virtually any dicot or monocot species can be stably transformed, and these cells can be regenerated into transgenic plants, through the application of the techniques disclosed herein. The plant cells, plants, and seeds can therefore be monocotyledons or dicotyledons.

The cell(s) that undergo transformation may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods available to those of skill in the art. Examples include, but are not limited to, transformation by direct DNA transfer into plant cells by electroporation (U.S. Pat. Nos. 5,384,253; 5,472,869; and Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. Nos. 5,489,520; 5,538,877; and 5,538,880) and DNA transfer to plant cells via infection with Agrobacterium. Methods, such as microprojectile bombardment or electroporation, can be carried out with “naked” DNA where the expression cassette may be simply carried on any E. coli-derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf disk protocol (Horsch et al., Science 227:1229 1231 (1985)). Monocots, such as Zea mays, can be transformed via microprojectile bombardment of embryogenic callus tissue or immature embryos or by electroporation following partial enzymatic degradation of the cell wall with a pectinase (U.S. Pat. Nos. 5,384,253; and 5,472,869). For example, embryogenic cell lines derived from immature Zea mays embryos can be transformed by accelerated particle treatment as described by Gordon Kamm et al. (The Plant Cell. 2:603 618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. Pat. No. 6,329,574 and Int'l Pat. App. Pub. No. WO 95/06128. Furthermore, methods for transformation of monocotyledonous plants utilizing Agrobacterium tumefaciens have been described by Hiei et al. (European Patent 0 604 662 (1994)) and Saito et al. (European Patent 0 672 752 (1995)).

Methods such as microprojectile bombardment or electroporation can be carried out with “naked” DNA where the expression cassette may be simply carried, for example, on any E. coli-derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are exemplary Zea mays tissue sources. Selection of tissue sources for transformation of monocots is described in detail in U.S. Pat. No. 6,329,574 and Int'l Pat. App. Pub. No. WO 95/06128, both of which are hereby specifically incorporated by reference for their teachings regarding same.

The transformation is carried out under conditions directed to the plant tissue of choice. The plant cells or tissue are/is exposed to the DNA or RNA carrying the targeting vector and/or other nucleic acids for an effective period of time. This may range from a less than one second pulse of electricity for electroporation to a 2-3 day co-cultivation in the presence of plasmid-bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, e.g., by mechanical wounding.

To effect transformation by electroporation, one may employ either friable tissues such as a suspension cell culture, or embryogenic callus, or alternatively, one may transform immature embryos or other organized tissues directly. The cell walls of the preselected cells or organs can be partially degraded by exposing them to pectin-degrading enzymes (pectinases or pectolyases) or by mechanical wounding in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the 0-glucuronidase or bar gene engineered for expression in maize. Bacteria were inactivated by ethanol dehydration prior to bombardment. A low level of transient expression of the 0-glucuronidase gene was observed 24-48 hours following DNA delivery. In addition, stable transformants containing the bar gene can be recovered following bombardment with either E. coli or Agrobacterium tumefaciens cells. It is contemplated that particles may contain DNA, rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a biolistic particle delivery system, which can be used to propel particles coated with DNA or cells through a screen, such as a stainless steel or Nytex screen, onto a filter surface covered with maize cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth here in one may obtain up to 1,000 or more foci of cells transiently expressing a marker gene. The number of cells in a focus, which expresses the exogenous gene product 48 hours post-bombardment, often ranges from about 1 to 10 and averages about 1 to 3.

In bombardment transformation, one may optimize the pre-bombardment culturing conditions and the bombardment parameters to yield the maximum number of stable transformants. Both the physical and biological parameters for bombardment can influence transformation frequency. Physical factors are those that involve manipulating the DNA/microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to optimize fully the conditions and/or to adjust physical parameters, such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants and/or plant cells that can be modified as described herein include alfalfa (e.g., forage legume alfalfa), algae, avocado, barley, broccoli, Brussels sprouts, cabbage, canola, cassava, cauliflower, cole vegetables, collards, corn, crucifers, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, and wheat. In some embodiments, the plant is a Brassicaceae or other Solanaceae species. In some embodiments, the plant or cell can be a maize plant or cell. In some embodiments, the plant is not a species of Arabidopsis, for example, in some embodiments, the plant is not Arabidopsis thaliana.

An exemplary embodiment of methods for identifying transformed cells involves exposing the bombarded cultures to a selective agent, such as a metabolic inhibitor, an antibiotic, an herbicide or the like. Cells, which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including, but not limited to, filters and solid culture medium. Bialaphos and glyphosate are provided as examples of agents suitable for selection of transformants, but the technique of this disclosure is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light, which can be detected on photographic or X-ray film, in a luminometer (or liquid scintillation counter), by devices that enhance night vision, or by a highly light-sensitive video camera, such as a photon-counting camera. All these assays are nondestructive, and transformed cells may be cultured further following identification. The photon-counting camera is especially valuable as it allows one to identify specific cells or groups of cells, which are expressing luciferase, and manipulate those in real time.

It is further contemplated that combinations of screenable and selectable markers may be useful for identification of transformed cells. For example, selection with a growth-inhibiting compound, such as bialaphos or glyphosate, at concentrations below those that cause 100% inhibition followed by screening of growing tissue for expression of a screenable marker gene, such as luciferase, would allow one to recover transformants from cell or tissue types that are not amenable to selection alone. In an illustrative embodiment embryogenic Type II callus of Zea mays L. can be selected with sub-lethal levels of bialaphos. Slowly growing tissue is subsequently screened for expression of the luciferase gene, and transformants can be identified.

### Regeneration and Seed Production

Cells that survive the exposure to the selective agent, or cells that have been scored positive in a screening assay, are cultured in medium that supports regeneration of plants. One example of a growth regulator that can be used for such purposes is dicamba or 2,4-D. However, other growth regulators may be employed, including NAA, NAA+2,4-D, or picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or a greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to have the mutations. In some embodiments, the regenerated plants are self-pollinated. In addition, pollen obtained from the regenerated plants can be crossed to seed-grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants to introgress the mutations into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced TFs, inactivating mutations of TFs, or expression cassette, the plant is self-pollinated at least once to produce a homozygous backcross-converted inbred containing the mutations. Progeny of these plants are true breeding.

Alternatively, seed from transformed mutant plant lines regenerated from transformed tissue cultures is grown in the field and self-pollinated to generate true breeding plants.

Seed from the fertile transgenic plants can then be evaluated for the presence of the desired TFs, inactivating mutations of TFs, the expression cassette, and/or the expression of the desired mutant protein. Transgenic plant and/or seed tissue can be analyzed using standard methods, such as SDS polyacrylamide gel electrophoresis, liquid chromatography (e.g., HPLC) or other means of detecting a mutation.

Once a transgenic plant with a mutant sequence and having improved lipid content, for example, is identified, seeds from such plants can be used to develop true breeding plants. The true breeding plants are used to develop a line of plants with increased lipid content, for example, relative to wild-type and acceptable growth characteristics, while still maintaining other desirable functional agronomic traits. Adding the mutation to other plants can be accomplished by back-crossing with this trait and with plants that do not exhibit this trait and studying the pattern of inheritance in segregating generations. Those plants expressing the target trait (insect resistance, good growth) in a dominant fashion are preferably selected. Back-crossing is carried out by crossing the original fertile transgenic plants with a plant from an inbred line exhibiting desirable functional agronomic characteristics, while not necessarily expressing the trait of an increased insect resistance and good plant growth. The resulting progeny are then crossed back to the parent that expresses increased lipid content and good plant growth. The progeny from this cross will also segregate so that some of the progeny carry the trait and some do not. This back-crossing is repeated until an inbred line with the desirable functional agronomic traits, and with expression of the trait involving an increase in lipid content, for example, and good plant growth, is obtained. Increased lipid content and good plant growth can be expressed in a dominant fashion.

The new transgenic plants can also be evaluated for a battery of functional agronomic characteristics, such as growth, lodging, kernel hardness, yield, resistance to disease and insect pests, drought resistance, and/or herbicide resistance.

Plants that may be improved by these methods include, but are not limited to, agricultural plants of all types, oil and/or starch plants (canola, potatoes, lupins, sunflower and cottonseed), forage plants (alfalfa, clover and fescue), grains (maize, wheat, barley, oats, rice, sorghum, millet and rye), grasses (switchgrass, prairie grass, wheat grass, sudangrass, sorghum, and straw-producing plants), and softwood, hardwood and other woody plants (e.g., those used for paper production such as poplar species, pine species, and eucalyptus). In some embodiments the plant is a gymnosperm. Examples of plants useful for pulp and paper production include most pine species, such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn and grasses (e.g., miscanthus, switchgrass, and the like) as well as trees, such as poplar, aspen, willow, and the like. Plants useful for generating dairy forage include legumes, such as alfalfa, as well as forage grasses, such as bromegrass and bluestem.

### Determination of Stably Transformed Plant Tissues

To confirm the presence of TFs, inactivating mutants of the TFs, or expression cassette in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced TFs or inactivating mutants of the TFs or of RNA expressed from an introduced expression cassette. For example, PCR also be used to reverse transcribe RNA into DNA, using enzymes such as reverse transcriptase, and then this DNA can be amplified through the use of conventional PCR techniques.

For example, if no amplification of TFs or inactivating mutants of the TFs mRNAs is observed, then a deletion mutation has successfully been introduced.

Information about mutations can also be obtained by primer extension or single nucleotide polymorphism (SNP) analysis.

Further information about the nature of the RNA product may be obtained by Northern blotting. This technique will demonstrate the presence of an RNA species and give information about the integrity of that RNA. The presence of some mutations can be detected by Northern blotting. The presence or absence of an RNA species (e.g., TF RNA) can also be determined using dot or slot blot Northern hybridizations. These techniques are modifications of Northern blotting and also demonstrate the presence or absence of an RNA species.

While Southern blotting and PCR may be used to detect the presence of TFs or inactivating mutants of the TFs or the presence of the expression cassette, they do not provide information as to whether the preselected DNA segment is being expressed. Expression may be evaluated by specifically identifying the protein products of the introduced expression cassette or the inactivating mutants of the TFs or evaluating the phenotypic changes brought about by such mutation.

Assays for the production and identification of specific proteins may make use of physical chemical, structural, functional, or other properties of the proteins. Unique physical chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to confirm the identity of a mutation such as evaluation by screening for reduced transcription (or no transcription) of TF inactivating mutant mRNAs, by screening for TF expression, or by amino acid sequencing following purification. The Examples of this application also provide assay procedures for detecting the TFs or inactivating mutants of the TFs or evaluating the seed oil in the resulting plants. Other procedures may be additionally used.

### EXAMPLES

The following examples serve to illustrate the present disclosure. The examples are not intended to limit the scope of the claimed invention in any way.

### Example 1: Methods

A. Arabidopsis thaliana Expressing Mutant TFs.

Arabidopsis thaliana expressing genes for inactivating mutants of each of the lipid-regulating transcription factors MYBS2, ATHB25, CESTA1, and bHLH093, was produced. Each inactivating mutant had the lipid-regulating transcription factor either knocked-out or knocked-down due to the insertion of a transposable element into the coding region of that gene in the genome. The mutant lines of Arabidopsis thaliana were grown in parallel to the control Arabidopsis genotype (Col-0) and the seeds derived from all plants were subjected to a lipid content assay (Fatty Acid Methyl Ester (FAME) in g per mg of seeds). The resulting lipid content measures for each genotype were compared against the Col-0 genotype to ascertain if the total lipid content was significantly altered in the mutant.

MYBS2 mutant: Mutant allele ID: SALK_206518; This genomic-terminal sequence of a TDNA insertion region lies within the mRNA region of AT5G08520. (chr 5 pos 2756158.5 R-SALK_206518 Env #: 74179). Insertion flanking sequence:

[SEQ ID NO: 17]
TTTGTTTATGCCACACAAGTATACAGAACCCTACTTCTAAATTGCTTCT
CCACTGGATTTTAATCTCCTAACTAAGCAGGAAAGACATTTCTAAACAC
CCGTACAACGTATTTGAAAATGAACCTCGTGATTTCTTGTCCAGGAGGT
TCTTCTATCACCGCCAAAATCCAATTTCTCGAAAAAGCACATGTAAACC
ACCAAATTATCAAGATGACACGGGAGTAGATCAAGATAATGGCGAAAAC
ACAAGTTTATAAAAATGGTGTAAGAATCT (Length: 274)

ATHB25 mutant: Mutant allele ID: SAILseq_517_E03.0; This genomic-terminal sequence of a TDNA insertion region lies within the CDS of AT5G65410.1 (at chr 5 pos 26136870 (W/26136790-26136870) on the TAIR10). The sequences' 1-81 bps mapped on the genome, 74-95 bps onto the T-DNA. Pools: P 11, R 14, C 13 N 13. The insertion site is at the 3′ end of the sequence mapping. Insertion flanking sequence:

[SEQ ID NO: 18]
AAGGATGTTAGCTTTAGCTGAGGGGATTGGATGGAGAAGTCAGAGACAA
GACGATGAAGTGATTCAGAGATTTTGTCAGGATATATTGTGGTGTA
(Length: 95)

CESTA1 mutant: Mutant allele ID: SAILseq_517_E03.0; This genomic-terminal sequence of a TDNA insertion region lies within the CDS of AT5G65410.1 (at chr 5 pos 26136870 (W/26136790-26136870) on the TAIR10). The sequences' 1-81 bps mapped on the genome, 74-95 bps onto the T-DNA. Pools: P 11, R 14, C 13 N 13. The insertion site is at the 3′ end of the sequence mapping. Insertion flanking sequence:

[SEQ ID NO: 19]
AAGGATGTTAGCTTTAGCTGAGGGGATTGGATGGAGAAGTCAGAGACAA
GACGATGAAGTGATTCAGAGATTTTGTCAGGATATATTGTGGTGTA
(Length: 95)

bHLH093 mutant: Mutant allele ID: SALKseq_121082.0; This genomic-terminal sequence of a TDNA insertion region lies within the CDS of AT5G65640.1 (at chr 5 pos 26237716 (W/26237685-26237716) on the TAIR10). The sequences' 1-32 bps mapped on the genome, 32-56 bps onto the T-DNA. Pools: P 51, R 55, C 55 N 59. The insertion site is at the 3′ end of the sequence mapping. Insertion flanking sequence:

[SEQ ID NO: 20]
GAGACGACGGAGAAAACGACTTAACGATCGTCAGATTGACGCTTAGACA
ACTTAATGTTAGCAGATCGGAAGAGCGGTT (Length: 79)

### B. Fatty Acid Methyl Ester (FAME) Assay

FAME assays were conducted according to procedures disclosed in A Rapid Method of Total Lipid Extraction and Purification. Bligh & Dyer, Canadian Journal of Biochemistry and Physiology, Vol. 37 (8), 1959, which is incorporated by reference.

### Example 2: Total Fatty Acid Content of Arabidopsis thaliana Seeds Expressing Mutant Lipid Regulating Transcription Factors

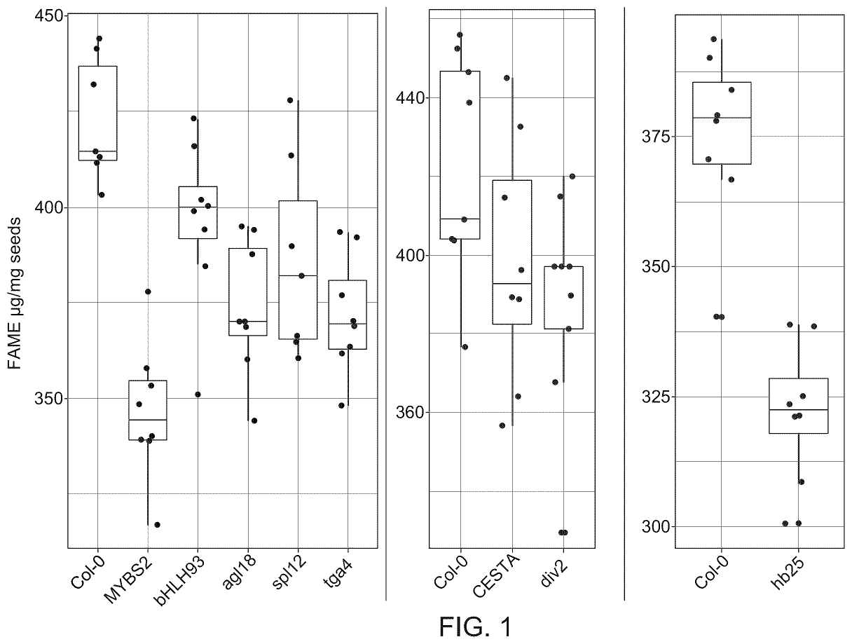

The FAME analyses of mutant lines confirmed that under-expression of any of the eight novel TFs reduces the lipid content of seeds (p-val <0.01, FIG. 1, FIG. 5). Under-expression of bHLH93 reduced the total lipid content by ˜10.5% relative to the control plants. Shown here are three panels, representing three batches in which these genes were evaluated. In each panel, the reference genotype Col-0 serves as the control against which the FAME content of mutant line is evaluated. Under-expression of any of the eight TFs listed in this disclosure leads to significant reduction in total lipid content in the seed (Student's t-test, p-val <0.01).

Similarly, under-expression of MYBS2 reduced lipid content by ˜18%; HB25 by ˜14.2%; CESTA by ˜7.9%; TGA4 by ˜12%; SPL12 by ˜11%; AGL18 by ˜11.6% and DiV2 by ˜7.2% (FIG. 1, FIG. 5). In each case the reduction of lipid content by under-expression of the specific TF listed was confirmed by a second, independent mutant line that also under-expresses the TF. Therefore, the under-expression of any of these eight TFs through genetic transformation is a viable approach to reduce lipid content in seeds. Further, the functionally equivalent version of each of these TFs (i.e., ortholog) can be readily identified in any oil crop species through homology sequence searches (e.g., BLAST search). The approach described here of under-expressing one or more of these specific TFs can then be applied in all oil crop species (e.g., canola, rape seed, camelina, soybean, corn, sunflower, cotton, avocado, etc.) by generating lines that lack or under-express the orthologs for these TFs.

### Example 3: Total Fatty Acid Content of Arabidopsis thaliana Seeds Over-Expressing Lipid-Regulating Transcription Factors

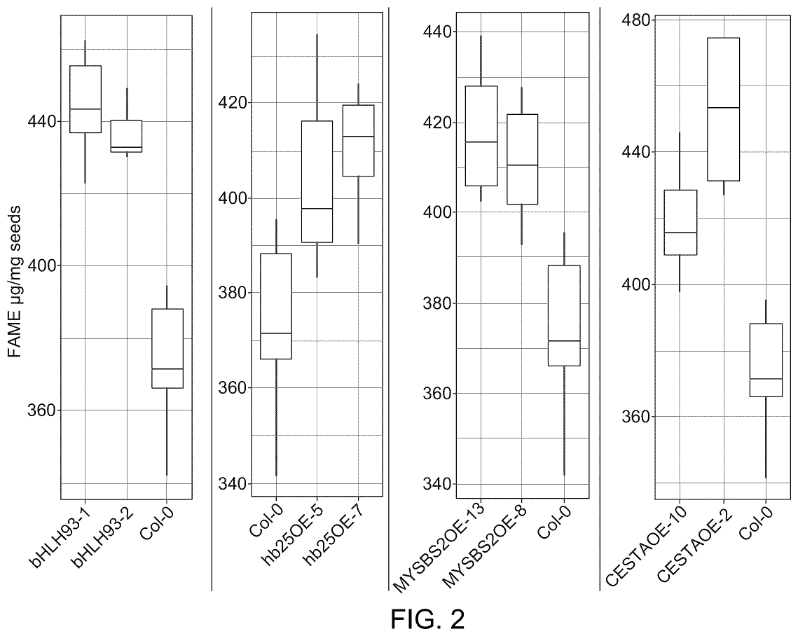

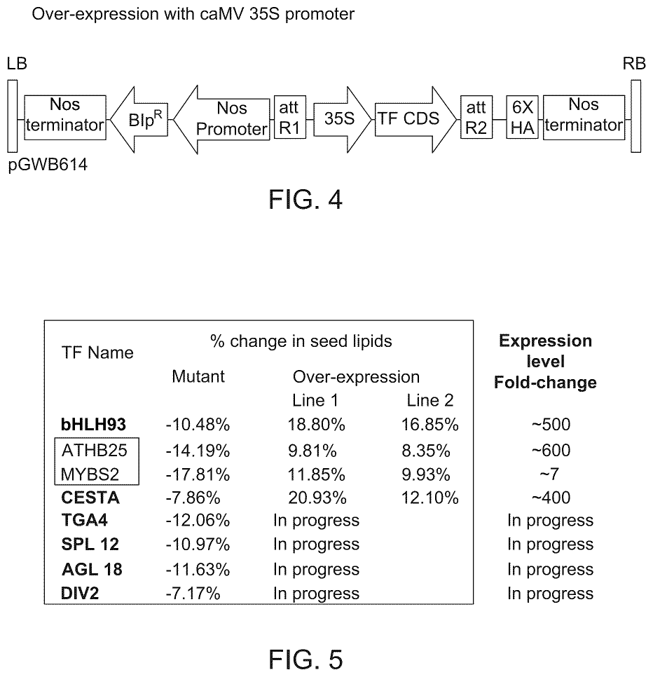

The effect of over-expressing each TF on seed lipid content was evaluated by generating stable transgenic lines that over-express the TF (FIG. 2, FIG. 5). Each transgenic line was created by transforming a Col-0 genotype plant with a construct (FIG. 4) that includes the specific TF fused to a constitutive 35S promoter. The level of expression of the targeted TF was measured in the transgenic line via a qRT-PCR assay to confirm the over-expression of TF. Four specific examples are shown where over-expression of these TFs increases the total lipid content of seeds. In the first example, over-expression of bHLH93 increased seed lipid content by either 18.8% or 16.85% in two independent genetic lines that both show approximately 500× higher levels of TF expression relative to the untransformed parent line (Col-0) under similar growth conditions. In a second example, over-expression of At5g08520 increased seed lipid content by 11.85% and 9.93% respectively in two independent genetic lines that express 7× higher levels of this TF. In a third example, over-expression of HB25 increased seed lipid content by 9.81% and 8.35% respectively in two independent genetic lines that express 600× higher levels of this TF. In a fourth example, over-expression of CESTA increased seed lipid content by 20.93% and 12.10% respectively in two independent genetic lines that express 400× higher levels of this TF (FIG. 5). Overall, the population of plants having an expression system expressing one of MYBS2, bHLH093, ATHB25, or CESTA1 has seeds with approximately 10% to approximately 20% more seed lipid content than a corresponding wild-type population of plants of the same age, wherein the wild-type population of plants does not have the expression system.

### Example 4: Total Fatty Acid Content of Arabidopsis thaliana Seeds Expressing Mutant Mybs2 Genes

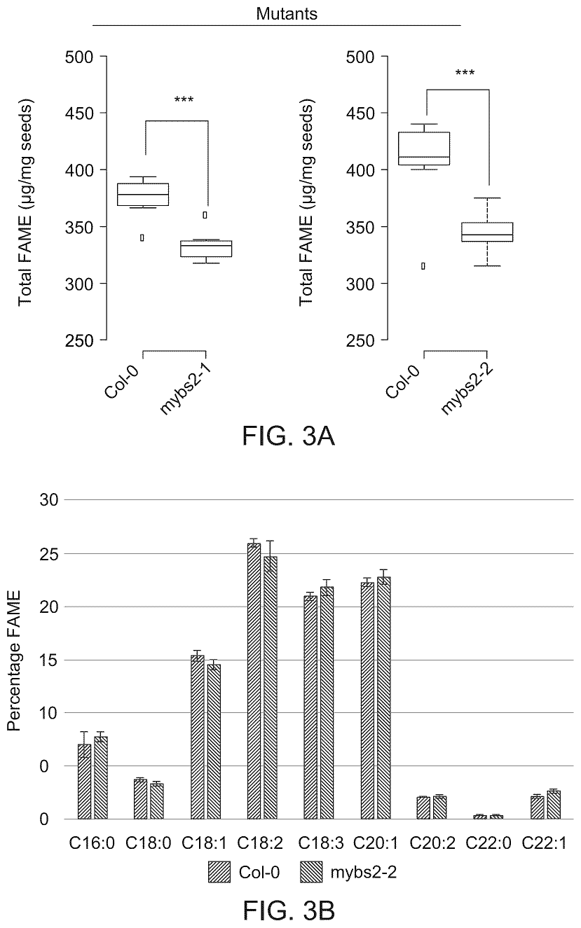

Two separate Arabidopsis thaliana mutant plant lines expressing two different mybs2 mutants were grown and total fatty acid content of their seeds was measured as Fatty Acid Methyl Ester (FAME) in g/mg of seeds (FIG. 3A). In each mutant, the amount total fatty acids was significantly reduced compared wild type Arabidopsis thaliana (Col-0). The composition of fatty acids in one of the mybs2 mutants and wild-type Arabidopsis thaliana was also determined (FIG. 3b). There was no significant difference in fatty acid composition between the two groups.

### Example 5: Summary of Seed Lipid Content Changes Caused by Altering Expression of Lipid-Regulating Transcription Factors

A summary of individual genetic strategies to alter lipid content and the expected change in lipid content is listed in the table of FIG. 5. A combination of these TFs can be over or under expressed to further increase or decrease the seed lipid content. Further, this method is applicable to any plant. For example, the plant may be an oil crop or fruit or vegetable species. This method is applicable to a broad range of plant species. For example, the plant species may be a monocot (e.g., corn, rice, sorghum etc.) or a dicot (rape seed, canola, camelina, cotton, soybean, grape, avocado etc.). The TF or combination of TFs can be linked to a promoter with functional activity in plants. For example, a constitutive promoter such as 35S, Ubiquitin etc. or a tissue specific promoter such as NapA can be used to drive the general or targeted expression of these TFs.

### Example 6: Over-Expression of Transcription Factors Increases Oil Content in Seeds

Arabidopsis transgenic lines constitutively over-expressing transcription factors were generated, and the total oil content in their seeds was analyzed. The following describes detailed methodologies used to generate TF overexpressing plants and estimate seed oil content.

### Molecular Cloning, Plasmid Preparation, and Plant Transformation

The coding DNA Sequence (CDS) of the MYBS2, CESTA, bHLH93, HB25, AGL18, and SPL12 transcription factor (TF) gene was amplified by specific primer pairs. The overexpression construct was prepared by cloning each CDS into the pENTR-D/TOPO vector (Invitrogen) and then mobilized to the binary vector pGWB614 (RIKEN, Japan) using Gateway™ LR Clonase™ II Enzyme mix (Thermo-Fisher Scientific). The construct will allow constitutive expression of the TF under the Cauliflower Mosaic Virus 35s (CaMV 35s) promoter and contain the BAR gene, which will provide resistance to glufosinate ammonium (BASTA) for the selection of transgenic plants (FIG. 4). Sanger sequencing was used to confirm the orientation and frame of the insert. 35S::MybS2-6HA construct was transformed into the Arabidopsis Col-0 plant through the Agrobacterium (strain GV3101) using the floral dip method. Transformants (T1) were screened by spraying BASTA (glufosinate ammonium, 0.01%) and PCR. Single insertion and T3-homozygous lines were obtained by analyzing the segregation pattern of the BASTA resistance gene. Several homozygous independent overexpression transgenic lines of each TF were used for the seed oil content estimation.

### Seed Oil Extraction and Quantification

Transgenic TF overexpressing and Col-0 control Arabidopsis plants were planted into the 4″ pot filled with standard germination mix (BM2) and turface (3:1) and grown in a growth room with a temperature of 21° C./18° C. day/night under long-day (16 h/8 h light/dark) condition of light intensity 150 umol/m²/sec. Mature seeds were harvested from each plant and desiccated for one week before any phenotypic analysis.

For the seed oil estimation, 10 mg of seeds were transmethylated in a glass vial at 90° C. for 90 min in 0.3 ml of toluene and 1 ml of 5% H2SO4 (v/v methanol). Each sample was dosed with 100 g of Heptadecanoic acid (C17:0) to serve as a non-native internal standard. After transmethylation, 1.5 ml of 0.9% NaCl solution was added, and extraction was performed using 2 ml of n-hexane. Fatty Acid Methyl Esters (FAMEs) were analyzed using TriPlus RSH autosampler and Trace 1310 gas chromatography (GC) system having a 50 m×0.25 mm FAME GC column of film thickness 0.25 um (Agilent Technologies, Santa Clara, CA) and coupled to a TSQ 8000 mass spectrometer (MS) (Thermo Fisher Scientific, Waltham, MA). A. The GC carrier gas was helium with a 1.0 ml/min linear flow rate. The programmed GC temperature gradient was as follows: time 0 minutes, 80° C. then ramped to 175° C. at a rate of 13° C./minute with a 5-minute hold, then ramped to 245° C. at a rate of 4° C./minute with a 2 minute at the end. The GC inlet was set to 250° C., and samples were injected in split mode using a split ratio of 20. The MS transfer line was set to 250° C., and the MS ion source was set to 250° C. and used EI ionization with 70 eV. MS data were collected in scanning mode with a 50-500 amu range. All data were analyzed with Thermo Fisher Chromeleon (Version 7.2.9) software, and quantities of each FA class were estimated after normalizing to the introduced C17:0 peak area. At least three independent biological replicates were used for each measurement, and a student t-test was used for significance evaluation.

### Results

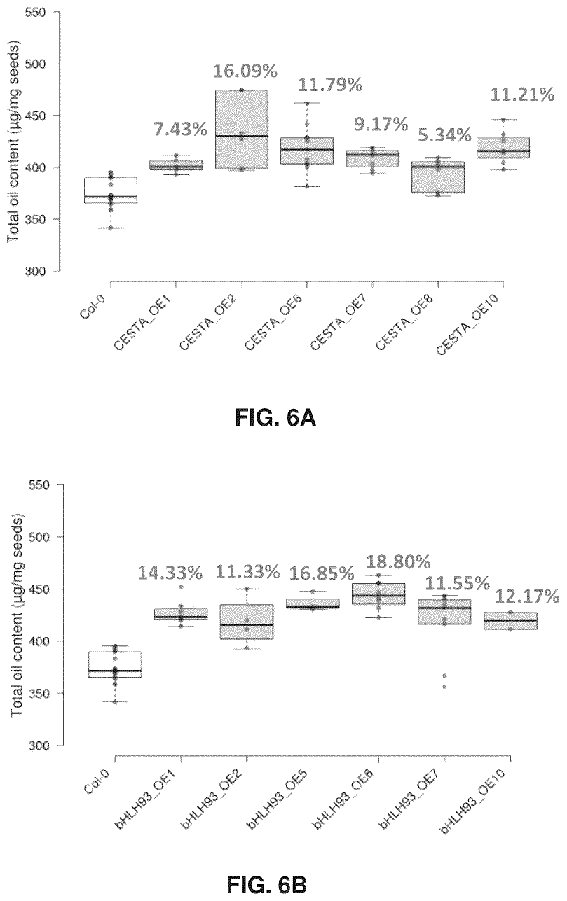

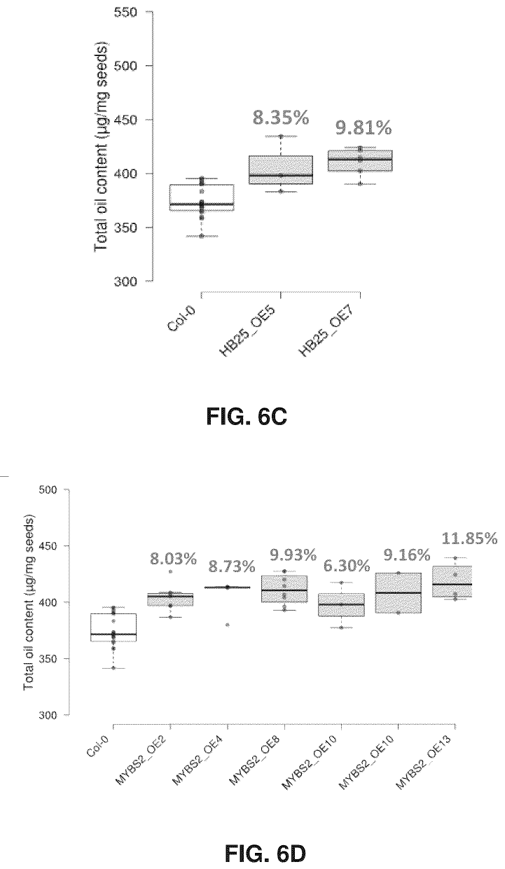

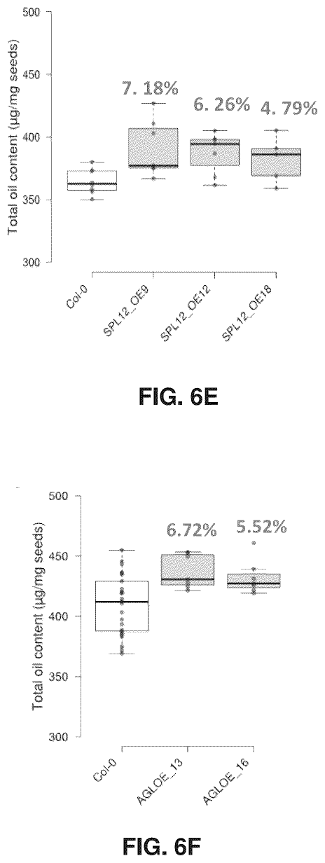

The effect of independent overexpression of transcription factors MYBS2, CESTA, bHLH93, HB25, AGL18, and SPL12 on total seed oil content was examined. Overexpression of MYBS2 resulted in an increase in total oil content from 6.3 to 11.85% in the seeds of six independent transgenic lines. Similarly, overexpression of bHLH93 and CESTA increased total seed oil in six independent lines with a range of 11.33%-18.80% and 5.43%-16.09%, respectively. Overexpression of HB25 and AGL18 caused increased seed oil in two independent transgenic lines with a range of 8.35%-9.81% and 5.52%-6.72%, respectively, and overexpression of SPL12 led to higher accumulation of total oil from 4.79-7.18% in three independent transgenic lines (FIGS. 6A-F).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to incorporate physically into this specification any and all materials and information from any such cited patents or publications.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

### REFERENCES

bHLH93
1. Poirier, B. C., M. J. Feldman, and B. M. Lange. 2018. “bHLH093/NFL and bHLH061 Are Required for Apical Meristem Function in Arabidopsis Thaliana.” Plant Signaling & Behavior 13 (7): e1486146.

### CESTA

2. Albertos, Pablo, Tanja Wlk, Jayne Griffiths, Maria J. Pimenta Lange, Simon J. Unterholzner, Wilfried Rozhon, Theo Lange, Alexander M. Jones, and Brigitte Poppenberger. 2022. “Brassinosteroid-Regulated bHLH Transcription Factor CESTA Induces the Gibberellin 2-Oxidase GA2ox7.” Plant Physiology 188 (4): 2012-25.
3. Crawford, Brian C. W., and Martin F. Yanofsky. 2011. “HALF FILLED Promotes Reproductive Tract Development and Fertilization Efficiency in Arabidopsis Thaliana.” Development 138 (14): 2999-3009.
4. Khan, Mamoona, Wilfried Rozhon, Simon Josef Unterholzner, Tingting Chen, Marina Eremina, Bernhard Wurzinger, Andreas Bachmair, et al. 2014. “Interplay between Phosphorylation and SUMOylation Events Determines CESTA Protein Fate in Brassinosteroid Signalling.” Nature Communications 5 (August): 4687.
5. Poppenberger, Brigitte, Wilfried Rozhon, Mamoona Khan, Sigrid Husar, Gerhard Adam, Christian Luschnig, Shozo Fujioka, and Tobias Sieberer. 2011. “CESTA, a Positive Regulator of Brassinosteroid Biosynthesis.” The EMBO Journal 30 (6): 1149-61.
6. Di Marzo, Maurizio, Irma Roig-Villanova, Eva Zanchetti, Francesca Caselli, Veronica Gregis, Paola Bardetti, Matteo Chiara, et al. 2020. “MADS-Box and bHLH Transcription Factors Coordinate Transmitting Tract Development in Arabidopsis Thaliana.” Frontiers in Plant Science 11 (May): 526.

### TGA4

7. Alvarez, José M., Eleodoro Riveras, Elena A. Vidal, Diana E. Gras, Orlando Contreras-López, Karem P. Tamayo, Felipe Aceituno, et al. 2014. “Systems Approach Identifies TGA1 and TGA4 Transcription Factors as Important Regulatory Components of the Nitrate Response of Arabidopsis Thaliana Roots.” The Plant Journal: For Cell and Molecular Biology 80 (1): 1-13.
8. Qi, Peipei, Mengling Huang, Xuehan Hu, Ying Zhang, Ying Wang, Pengyue Li, Shiyun Chen, et al. 2022. “A Ralstonia Solanacearum Effector Targets TGA Transcription Factors to Subvert Salicylic Acid Signaling.” The Plant Cell 34 (5): 1666-83.

### FAME Assay

19. Bligh, E. G., and W. J. Dyer. 1959. “A Rapid Method of Total Lipid Extraction and Purification.” Canadian Journal of Biochemistry and Physiology 37 (8): 911-17.

### HB25

20. Bueso, Eduardo, Jesus Munoz-Bertomeu, Francisco Campos, Veronique Brunaud, Liliam Martinez, Enric Sayas, Patricia Ballester, Lynne Yenush, and Ram6n Serrano. 2014. “ARABIDOPSIS THALIANA HOMEOBOX25 Uncovers a Role for Gibberellins in Seed Longevity.” Plant Physiology 164 (2): 999-1010.
21. Renard, Joan, Irene Martinez-Almonacid, Indira Queralta Castillo, Annika Sonntag, Aseel Hashim, Gaetano Bissoli, Laura Campos, et al. 2021. “Apoplastic Lipid Barriers Regulated by Conserved Homeobox Transcription Factors Extend Seed Longevity in Multiple Plant Species.” The New Phytologist 231 (2): 679-94.

### SPL12

22. Chao, Lu-Men, Yao-Qian Liu, Dian-Yang Chen, Xue-Yi Xue, Ying-Bo Mao, and Xiao-Ya Chen. 2017. “Arabidopsis Transcription Factors SPL1 and SPL12 Confer Plant Thermotolerance at Reproductive Stage.” Molecular Plant 10 (5): 735-48.

### MYBS2

23. Chen, Yi-Shih, Yi-Chi Chao, Tzu-Wei Tseng, Chun-Kai Huang, Pei-Ching Lo, and Chung-An Lu. 2017. “Two MYB-Related Transcription Factors Play Opposite Roles in Sugar Signaling in Arabidopsis.” Plant Molecular Biology 93 (3): 299-311.
24. Chen, Yi-Shih, Tuan-Hua David Ho, Lihong Liu, Ding Hua Lee, Chun-Hua Lee, Yi-Ru Chen, Shu-Yu Lin, Chung-An Lu, and Su-May Yu. 2019. “Sugar Starvation-Regulated MYBS2 and 14-3-3 Protein Interactions Enhance Plant Growth, Stress Tolerance, and Grain Weight in Rice.” Proceedings of the National Academy of Sciences of the United States of America 116 (43): 21925-35.
24. Wang, Ting, Takayuki Tohge, Alexander Ivakov, Bernd Mueller-Roeber, Alisdair R. Fernie, Marek Mutwil, Jos H. M. Schippers, and Staffan Persson. 2015. “Salt-Related MYB1 Coordinates Abscisic Acid Biosynthesis and Signaling during Salt Stress in Arabidopsis.” Plant Physiology 169 (2): 1027-41.

### AGL18

25. Paul, Priyanka, Sanjay Joshi, Ran Tian, Rubens Diogo Junior, Manohar Chakrabarti, and Sharyn E. Perry. 2022. “The MADS-Domain Factor AGAMOUS-Like18 Promotes Somatic Embryogenesis.” Plant Physiology 188 (3): 1617-31.
26. Serivichyaswat, Phanu, Hak-Seung Ryu, Wanhui Kim, Soonkap Kim, Kyung Sook Chung, Jae Joon Kim, and Ji Hoon Ahn. 2015. “Expression of the Floral Repressor miRNA156 Is Positively Regulated by the AGAMOUS-like Proteins AGL15 and AGL18.” Molecules and Cells 38 (3): 259-66.
27. Zheng, Qiaolin, and Sharyn E. Perry. 2014. “Alterations in the Transcriptome of Soybean in Response to Enhanced Somatic Embryogenesis Promoted by Orthologs of Agamous-like15 and Agamous-like18.” Plant Physiology 164 (3): 1365-77.
28. Thakare, Dhiraj, Weining Tang, Kristine Hill, and Sharyn E. Perry. 2008. “The MADS-Domain Transcriptional Regulator AGAMOUS-LIKE15 Promotes Somatic Embryo Development in Arabidopsis and Soybean.” Plant Physiology 146 (4): 1663-72.

### DiV2

29. Fang, Qing, Qiong Wang, Hui Mao, Jing Xu, Ying Wang, Hao Hu, Shuai He, et al. 2018. “AtDIV2, an R—R-Type MYB Transcription Factor of Arabidopsis, Negatively Regulates Salt Stress by Modulating ABA Signaling.” Plant Cell Reports 37 (11): 1499-1511.

### Enumerated Embodiments

1. A plant cell, plant seed, or plant comprising an expression system comprising an expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

2. The plant cell, plant seed, or plant of embodiment 1, wherein the plant seed has approximately 10% to approximately 20% more seed lipid content than a corresponding wild-type plant seed.

3. The plant cell, plant seed, or plant of embodiment 1, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

4. The plant cell, plant seed, or plant of embodiment 3, wherein the nucleic acid segment encodes an inactivating mutant of the polypeptide for MYBS2, bHLH093, ATHB25, DiV2, CESTA1, TGA4, SPL12, or AGL18.

5. The plant cell, plant seed, or plant of embodiment 4, wherein the plant seed has approximately 7.2% to approximately 18% less seed lipid content than a corresponding wild-type plant seed.

6. The plant cell, plant seed, or plant of embodiment 1, wherein the plant cell, plant seed, or plant is an oil crop plant cell, plant seed, or plant.

7. The plant cell, plant seed, or plant of embodiment 6, wherein the oil crop is a Brassica species, camelina, soybean, corn, sunflower, cotton, peanut, or avocado.

8. The plant cell, plant seed, or plant of embodiment 7, wherein the Brassica plant species is Brassica rapa, Brassica juncea, Brassica napus, or Brassica carinata.

9. The plant cell, plant seed, or plant of embodiment 1, wherein the promoter is a strong or inducible promoter.

10. The plant cell, plant seed, or plant of embodiment 1, wherein the promoter is a tissue-specific promoter.

11. The plant cell, plant seed, or plant of embodiment 1, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

12. An expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

13. The expression cassette of embodiment 12, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

14. The expression cassette of embodiment 12, wherein the promoter is a strong or inducible promoter.

15. The expression cassette of embodiment 12, wherein the promoter is a tissue-specific promoter.

16. The expression cassette of embodiment 12, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

17. A method of growing a plant seed or plant comprising:
introducing into at least one plant cell at least one transgene or expression cassette
encoding a polypeptide for MYBS2, bHLH093, ATHB25, DiV2, CESTA1, TGA4, SPL12, or AGL18, or a combination of two, three, four, five, six, seven or all thereof, to generate one or more transformed plant cells; and
generating a plant from the one or more transformed plant cell(s).

18. The method of embodiment 17, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

19. The method of embodiment 17, further comprising harvesting lipids from the seeds of the mature plant.

20. The method of embodiment 17, wherein the promoter is a strong or inducible promoter.

21. The method of embodiment 17, wherein the promoter is a tissue-specific promoter.

22. The method of embodiment 17, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

23. The method of embodiment 17, wherein the plant seed or plant is an oil crop.

24. The method of embodiment 23, wherein the oil crop is a Brassica plant species, camelina, soybean, corn, sunflower, cotton, peanuts, or avocado.

25. The method of embodiment 24, wherein the Brassica plant species is Brassica rapa, Brassica juncea, Brassica napus, or Brassica carinata.

26. The method of embodiment 17, wherein the nucleic acid segment encodes an inactivating mutant of the polypeptide for MYBS2, bHLH093, ATHB25, DiV2, CESTA1, TGA4, SPL12, or AGL18.

27. The method of embodiment 26, further comprising harvesting starch from the seeds of the mature plant.

What is claimed is:

1. A plant cell, plant seed, or plant comprising an expression system comprising an expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

2. The plant cell, plant seed, or plant of claim 1, wherein the plant seed has approximately 10% to approximately 20% more seed lipid content than a corresponding wild-type plant seed.

3. The plant cell, plant seed, or plant of claim 1, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

4. The plant cell, plant seed, or plant of claim 3, wherein the nucleic acid segment encodes an inactivating mutant of the polypeptide for MYBS2, bHLH093, ATHB25, DiV2, CESTA1, TGA4, SPL12, or AGL18.

5. The plant cell, plant seed, or plant of claim 4, wherein the plant seed has approximately 7.2% to approximately 18% less seed lipid content than a corresponding wild-type plant seed.

6. The plant cell, plant seed, or plant of claim 1, wherein the plant cell, plant seed, or plant is an oil crop plant cell, plant seed, or plant.

7. The plant cell, plant seed, or plant of claim 6, wherein the oil crop is a Brassica species, camelina, soybean, corn, sunflower, cotton, peanut, or avocado.

8. The plant cell, plant seed, or plant of claim 7, wherein the Brassica plant species is Brassica rapa, Brassica juncea, Brassica napus, or Brassica carinata.

9. The plant cell, plant seed, or plant of claim 1, wherein the promoter is a strong or inducible promoter.

10. The plant cell, plant seed, or plant of claim 1, wherein the promoter is a tissue-specific promoter.

11. The plant cell, plant seed, or plant of claim 1, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

12. An expression cassette comprising a promoter operably linked to a nucleic acid segment encoding a polypeptide for MYBS2, bHLH093, ATHB25, DiV2, CESTA1, TGA4, SPL12, or AGL18, or a combination of two, three, four, five, six, seven or all thereof.

13. The expression cassette of claim 12, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

14. The expression cassette of claim 12, wherein the promoter is a strong or inducible promoter.

15. The expression cassette of claim 12, wherein the promoter is a tissue-specific promoter.

16. The expression cassette of claim 12, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

17. A method of growing a plant seed or plant comprising:
introducing into at least one plant cell at least one transgene or expression cassette encoding a polypeptide for MYBS2, bHLH093, ATHB25, DiV2, CESTA1, TGA4, SPL12, or AGL18, or a combination of two, three, four, five, six, seven or all thereof, to generate one or more transformed plant cells; and
generating a plant from the one or more transformed plant cell(s).

18. The method of claim 17, wherein:
the polypeptide for MYBS2 has at least 95% sequence identity to SEQ ID NO: 1;
the polypeptide for bHLH093 has at least 95% sequence identity to SEQ ID NO: 3;
the polypeptide for ATHB25 has at least 95% sequence identity to SEQ ID NO: 5;
the polypeptide for DiV2 has at least 95% sequence identity to SEQ ID NO: 7;
the polypeptide for CESTA1 has at least 95% sequence identity to SEQ ID NO: 9;
the polypeptide for TGA4 has at least 95% sequence identity to SEQ ID NO: 11;
the polypeptide for SPL12 has at least 95% sequence identity to SEQ ID NO: 13; and
the polypeptide for AGL18 has at least 95% sequence identity to SEQ ID NO: 15.

19. The method of claim 17, further comprising harvesting lipids from the seeds of the mature plant.

20. The method of claim 17, wherein the promoter is a strong or inducible promoter.

21. The method of claim 17, wherein the promoter is a tissue-specific promoter.

22. The method of claim 17, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter.

23. The method of claim 17, wherein the plant seed or plant is an oil crop.

24. The method of claim 23, wherein the oil crop is a Brassica plant species, camelina, soybean, corn, sunflower, cotton, peanuts, or avocado.

25. The method of claim 24, wherein the Brassica plant species is Brassica rapa, Brassica juncea, Brassica napus, or Brassica carinata.

26. The method of claim 17, wherein the nucleic acid segment encodes an inactivating mutant of the polypeptide for MYBS2, bHLH093, ATHB25, DiV2, CESTA1, TGA4, SPL12, or AGL18.

27. The method of claim 26, further comprising harvesting starch from the seeds of the mature plant.


SEQUENCE LISTING (20 sequences)

SEQ ID NO: 1 - protein, 298 aa, Arabidopsis thaliana
MTVEEVSDGSVWSREDDIAFERALANNTDESEERWEKIAADVPGKSVEQIKEHYELLVEDVTRIESGCVPLPAYGSPEGSNGHAGDEGASSKKGGNSHAGESNQAGKSKSDQERRKGIAWTEDEHRLFLLGLDKYGKGDWRSISRNFVVTRTPTQVASHAQKYFIRLNSMNKDRRRSSIHDITSVGNADVSTPQGPITGQNNSNNNNNNNNNNSSPAVAGGGNKSAKQAVSQAPPGPPMYGTPAIGQPAVGTPVNLPAPPHMAYGVHAAPVPGSVVPGAAMNIGQMPYTMPRTPTAHR

SEQ ID NO: 2 - DNA, 2022 bp, Arabidopsis thaliana
  source 1..2022: mol_type = genomic DNA
atgaggagtagcagtaataaaatacatatggcataatttggtcaacaaggaaattccaatatgaattggaagtgtaacaaataataaaagagtcaacaataccaaaaataacaacagcggacttgagctgtgaaaactgttgcttatggtttttattcactgtttctttgttttgaacttccccttcctttaacaatggcgttttgacccctccctatctctctctctctctctctcagtcttctgggtttttcctattcctctttctctctctctccttcaagttgctgcaatcccttgaaaacccaataaacccccaattttccatttctcataaagttcacatttttccttcttcttcttctgccaattctctgattccctcgtttcaatctccgttttgctttgcctatcagataaatttcttcttgctttcctttctatctcataacgataagttgaattaatctttgcgtcttagttcatcagtagagagagtagggttttttccgcttatttttagggaatttcattttgttggaggtgagaatctctattcgagtccccaagattctctttatatccctagtttagttgtatggttgggttttgattggaatatcaaggggtagtttttagctaggttcactgatacttggaagatctgagttctttgggattctgttaagtttgtggagatctaaaagacacgaaatttgtagaaatctggttgatatcccagacttttagagggattagggtagattctatagaatttgaggcgggtttgattggaattatgacggtggaggaagttagtgatggttctgtgtggagtagggaggatgatattgcctttgagagagctctagccaataataccgatgaatcagaggaacggtgggagaagattgctgcagacgttccaggcaaaagtgttgaacagattaaagaacattacgagcttttagttgaagatgttactaggattgaatcaggatgtgtgcctcttcctgcctatgggtctcctgaaggatcgaatggccatgctggtgatgaaggagcaagtagtaagaaaggaggtaacagtcatgcgggagagtctaaccaagcaggtaaatcaaagtccgatcaagaacgacgaaagggtatcgcgtggacagaagatgagcacaggttatttcttcttggtttggataagtacgggaaaggtgattggcgtagcatttctcgcaactttgtagtaacaagaacaccgacccaagttgcgagccatgctcaaaagtatttcattcgtctaaattcaatgaacaaagacagaaggcgatcaagcattcacgacatcactagtgttggcaacgcagatgtctcaacgccacaaggaccaatcactggtcagaacaacagcaataacaacaacaacaacaacaacaacaacagttctcctgctgttgctggaggaggaaacaaatcagccaagcaagccgtctctcaagcaccacctggacctcctatgtatggaacacccgccataggtcagccagcagttggaacaccagtgaacctcccagctccacctcacatggcttatggagttcatgcggctccagtccctggctcagtggttcctggtgcagcaatgaacattggtcaaatgccgtacaccatgccgcgtacaccaacggctcataggtaactcgaaagcacctttgctgtcatagtgcactttgtttttaggtgtaagaaagaagatgtgtaaaggatttagtgaatattcaagcttgttccttgagtgagttttttttattacttagtttgtggggattttgtatgaggtccgaataagatatgaagatgacatgattagtttccagactcgagaagcaaaaatactcctgtttgtatgtgaacacaataaagcctctgttatgagacttacaacaaagcaacattgtatatcttgttctcacattcaacaatctctttgaattatcaactgcaacgtgcaattccttattttga

SEQ ID NO: 3 - protein, 351 aa, Arabidopsis thaliana
MELSTQMNVFEELLVPTKQETTDNNINNLSFNGGFDHHHHQFFPNGYNIDYLCFNNEEEDENTLLYPSSFMDLISQPPPLLLHQPPPLQPLSPPLSSSATAGATFDYPFLEALQEIIDSSSSSPPLILQNGQEENFNNPMSYPSPLMESDQSKSFSVGYCGGETNKKKSKKLEGQPSKNLMAERRRRKRLNDRLSMLRSIVPKISKMDRTSILGDAIDYMKELLDKINKLQDEEQELGNSNNSHHSKLFGDLKDLNANEPLVRNSPKFEIDRRDEDTRVDICCSPKPGLLLSTVNTLETLGLEIEQCVISCFSDFSLQASCSEGAEQRDFITSEDIKQALFRNAGYGGSCL

SEQ ID NO: 4 - DNA, 1056 bp, Arabidopsis thaliana
  source 1..1056: mol_type = genomic DNA
atggaactgtcgactcaaatgaatgtgttcgaagagcttcttgttccgacaaagcaagaaacaaccgacaacaacatcaacaatctgagctttaatggcggatttgatcatcatcatcatcaattcttcccaaatggatataatattgattacctctgtttcaacaatgaagaagaggacgaaaatacccttttgtatccttcttctttcatggatctaatctctcaacctcctccattgcttcttcaccaaccgccaccgttacaaccactgtcgccgccgttatcctcctccgcgaccgccggagcaacatttgactacccttttcttgaggctttgcaagagataattgactcttcttcctcatcgcctccattgatccttcaaaatggtcaagaagagaactttaataatccgatgtcgtatccctctccattgatggagtctgatcagagcaagagcttcagtgttggttactgtggaggagagacgaacaagaagaagagcaaaaagcttgaaggccaaccttctaagaatctcatggcggagagacgacggagaaaacgacttaacgatcgtctttctatgctccgatccatcgtcccaaaaatcagtaagatggacaggacatcgatattaggagatgccatagattacatgaaagagcttttagacaaaatcaacaaattacaagatgaggaacaagaacttggaaatagcaacaattcacatcactctaagctcttcggtgatctcaaggatcttaatgcgaacgaacctctggtcagaaactcaccaaagtttgaaatagatcgtagagacgaggatactcgagttgatatatgctgctcgccaaaaccgggattgctactatctactgtgaatacattagagactctaggcttggagattgaacaatgtgttataagctgctttagtgatttctctttgcaggcttcttgttctgagggagctgagcagagagatttcataacatcagaagatataaaacaagcattattcagaaacgcaggttatggtggaagctgcttgtaa

SEQ ID NO: 5 - protein, 279 aa, Arabidopsis thaliana
MEFEDNNNNNDEEQEEDMNLHEEEEDDDAVYDSPPLSRVLPKASTESHETTGTTSTGGGGGFMVVHGGGGSRFRFRECLKNQAVNIGGHAVDGCGEFMPAGIEGTIDALKCAACGCHRNFHRKELPYFHHAPPQHQPPPPPPGFYRLPAPVSYRPPPSQAPPLQLALPPPQRERSEDPMETSSAEAGGGIRKRHRTKFTAEQKERMLALAERIGWRIQRQDDEVIQRFCQETGVPRQVLKVWLHNNKHTLGKSPSPLHHHQAPPPPPPQSSFHHEQDQP

SEQ ID NO: 6 - DNA, 1090 bp, Arabidopsis thaliana
  source 1..1090: mol_type = genomic DNA
acattatctctctcttcttcttcaccttcttgagaaacctcctctccccaaaaacgacgtagtttcacatttctcgacttcttgaatggagtttgaagacaacaacaacaacaacgacgaagagcaagaagaggatatgaatcttcatgaggaagaagaagacgacgacgccgtttacgactctcctcctctctctcgtgttctccccaaagcctcgacagaaagtcatgaaaccaccggaactacttccacaggcggtggcggaggattcatggttgttcacggcggtggagggagcaggtttaggttccgtgagtgtctcaagaaccaagcggtgaacataggaggacacgcggtcgatggttgtggtgagtttatgccagctggaatcgaaggtaccatcgacgctctaaaatgcgccgcttgtggctgtcaccgtaacttccaccgcaaggaattaccttacttccatcacgcgccgccacaacatcagcctcctcctcccccgccagggttttaccgtcttccagctccggttagctaccgaccaccaccgtcacaagctcctcctcttcagctcgctcttccccctccacaaagagagagatcagaagatccaatggagacgtcttcagctgaagcaggaggagggattaggaagaggcataggactaagtttacggctgagcaaaaggaaaggatgttagctttagctgagaggattggatggagaattcagagacaagacgatgaagtgattcagagattttgtcaggagactggtgttccgagacaagttcttaaggtttggttacataacaacaaacacactcttggtaagtcgccttcaccacttcatcatcatcaggctcctcctcctccaccaccacagtcttcgtttcatcatgaacaagaccaaccatgaatcttgaatttctttgatcactagggttttaatttagcttaattaattacttgagaaatttgagagacaaggtttttattgtttaatttatgtacccattttcctctttgatgatgatgttgatgatgttggtgatgatctttaatttctggttaattatgtttt

SEQ ID NO: 7 - protein, 215 aa, Arabidopsis thaliana
MASSQWTRSEDKMFEQALVLFPEGSPNRWERIADQLHKSAGEVREHYEVLVHDVFEIDSGRVDVPDYMDDSAAAAAGWDSAGQISFGSKHGESERKRGTPWTENEHKLFLIGLKRYGKGDWRSISRNVVVTRTPTQVASHAQKYFLRQNSVKKERKRSSIHDITTVDATLAMPGSNMDWTGQHGSPVQAPQQQQIMSEFGQQLNPGHFEDFGFRM

SEQ ID NO: 8 - DNA, 1128 bp, Arabidopsis thaliana
  source 1..1128: mol_type = genomic DNA
aactgagagaaagagagagacaaaagacatcccaattgcagagaaaccgtgttgaagttggtctctgagctgcttcttccttcccttttttatttttatttttccagctctttattttcttatttacaaaaaactttcccaagaataaaaaccagaagaatccgtataaattatcctaacagtttcttccaatatccaacaaatttcagatttttgtttttgttttcttcttctctacttgagtaatcatcaacgattatggcgtcaagtcagtggacgaggtcggaggataagatgtttgagcaagctttggttctttttcctgaaggatctcctaatcggtgggagagaatcgctgatcagcttcataaatctgctggtgaagttagggagcattacgaggtcttggttcatgatgttttcgagattgattctggtcgagttgatgtccctgattacatggatgactcggcggctgcggcggcgggttgggattccgctggtcagatctcttttgggtctaaacatggcgagagtgaacgcaaaagaggaactccttggacagagaacgaacacaaattgtttctgatcggattaaagagatatggtaagggagattggaggagtatctcgagaaacgttgtggtgacgaggacaccgacgcaagtcgcgagtcacgctcagaagtattttctgagacagaactcggtgaagaaggagaggaaaaggtcgagcatccatgatataactacggttgatgctactttggctatgcctgggtctaacatggactggactggccaacacgggagtcctgttcaggcgccgcagcagcaacagattatgtctgagttcggtcagcaattgaatcctggtcatttcgaggattttgggtttcggatgtgatgaagaaagggggagacaaattgggatggctttagtttaatagggttttactcattttatgtgaatagggaaagaaataggattgggtattttcttatacagatgatgatgatgatgatcaaagaataaaaataatgtatagggaagctttttgtaaacacaaatatgtttggtaaccattttggtattttgaatcaatgcatatggttgtttttctcgatt

SEQ ID NO: 9 - protein, 223 aa, Arabidopsis thaliana
MARFEPYNYNNGHDPFFAHINQNPELINLDLPASTPSSFMLFSNGALVDANHNNSHFFPNLLHGNTRRKGNKEESGSKRRRKRSEEEEAMNGDETQKPKDVVHVRAKRGQATDSHSLAERVRREKINERLKCLQDLVPGCYKAMGMAVMLDVIIDYVRSLQNQIEFLSMKLSAASACYDLNSLDIEPTDIFQGGNIHSAAEMERILRESVGTQPPNFSSTLPF

SEQ ID NO: 10 - DNA, 1002 bp, Arabidopsis thaliana
  source 1..1002: mol_type = genomic DNA
ataactgtcttagagaaagaaaaaaaacaaaactagctcacaaaaaggaaatcatattttgatttaatttgtagtgtctctaatggcacggtttgagccatataactataataatggtcatgatcctttctttgcacacattaaccaaaatccagagctaataaatctggacttaccagcttctaccccttccagtttcatgcttttctccaatggagctttagttgatgccaatcacaataattctcacttcttcccaaatttattgcacggtaatacgagaagaaaaggaaataaagaagagagtgggtcgaagagaagaagaaagaggtcggaagaggaagaagccatgaatggagatgagactcagaagccaaaagatgttgttcatgtccgagctaagagaggtcaagctactgatagccatagtttggctgaaagggtacgaagagagaagatcaatgaaaggctgaaatgcttacaagaccttgttccaggatgctacaaggcaatgggaatggcagtgatgcttgatgtcatcatagattatgtacgatcactccagaatcaaatcgagtttttgtccatgaaactctcagcggcaagtgcatgttacgaccttaattctttggatattgagccaacggatatatttcagggagggaatattcatagtgcagcagagatggaaaggattttaagagaaagcgttggaacacagcctcctaatttcagttcaacattacccttttgatcataagaaaattatgaattttcagagaaattattctctttttctataattaaactccataaataaggacttaccatgatcagtatatataggtttatctatcttttgtgtgtgacgtcagtatacttttatcataaatgtgtaaccttatgattatgaagcttatccatatagtatgtaccatgaaaatgagtaaagctatatgttacaaagaaactctatttgaagtaacataagatttcgatatt

SEQ ID NO: 11 - protein, 364 aa, Arabidopsis thaliana
MNTTSTHFVPPRRFEVYEPLNQIGMWEESFKNNGDMYTPGSIIIPTNEKPDSLSEDTSHGTEGTPHKFDQEASTSRHPDKIQRRLAQNREAARKSRLRKKAYVQQLETSRLKLIHLEQELDRARQQGFYVGNGVDTNALSFSDNMSSGIVAFEMEYGHWVEEQNRQICELRTVLHGQVSDIELRSLVENAMKHYFQLFRMKSAAAKIDVFYVMSGMWKTSAERFFLWIGGFRPSELLKVLLPHFDPLTDQQLLDVCNLRQSCQQAEDALSQGMEKLQHTLAESVAAGKLGEGSYIPQMTCAMERLEALVSFVNQADHLRHETLQQMHRILTTRQAARGLLALGEYFQRLRALSSSWAARQREPT

SEQ ID NO: 12 - DNA, 1966 bp, Arabidopsis thaliana
  source 1..1966: mol_type = genomic DNA
gagaaatacagaataaccagaaacaaaattaaattagtaatcgaataaattatggacttaaaggtatcttaaacatgtcaaaatttggtttttgactgtgtagctgatgtatctagtacatatacttaaaggacaaatatcaccgaagaatcaacaaaccaaaaaaaaaaacagaaataaatgttagtatatatatttttattgacatttattaggatatagaaaaatttagaaaactcaatcaaaggtctctcttttaaagttgtcgtgttctctcttgaatgattcttcttctccttcttcgagatgacaagttcagagaacgagattttaccatcccttattctatcagaccggtttaagctgcagatttctaggcttagcgttcgatttcgtcgctgaaagtgaaaagttcatctagctttagttttctcttttcatggtttccgcgggaaaagttcgtcttttttgaagcccttttgacacaaaagaccagaacaagttgaagaaatatgaatacaacctcgacacattttgttccaccgagaaggtttgaagtttacgagcctctcaaccaaatcggtatgtgggaagaaagtttcaagaacaatggagacatgtatacgcctggctctatcataatcccgactaacgaaaaaccagacagcttgtcagaggatacttctcatgggacagaaggaactcctcacaagtttgaccaagaggcttccacatctagacatcctgataagatacagagaaggctagcacagaatcgagaggcagctaggaaaagtcgtttgcgcaagaaagcttatgttcagcagctagagactagccggttaaagctaattcatttagagcaagaactcgatcgtgctagacaacagggtttctatgtggggaacggagtagataccaatgctcttagtttctcagataacatgagctcagggattgttgcatttgagatggaatatggacattgggtggaagaacagaacaggcaaatatgtgaactaagaacggttttacatggacaagttagtgatatagagcttcgttctctagtcgagaatgccatgaaacattactttcaactcttccgaatgaagtcagccgctgcaaaaatcgatgttttctatgtcatgtccggaatgtggaaaacttcagcagagcggtttttcttgtggataggcggatttagaccctcagagcttctcaaggttctgttaccgcattttgatcctttgacggatcaacaacttttggatgtatgtaatctgaggcaatcatgtcaacaagcagaagatgcgttatcccaaggtatggagaaactgcaacatacattagcagagagtgtagcagccgggaaacttggtgaaggaagttatattcctcaaatgacttgtgctatggagagattggaggctttggtcagctttgtaaatcaagctgatcatctgagacatgagacattgcaacagatgcatcggatcttaaccacgcgacaagcggctagaggtttgttagcattaggggagtatttccaaaggcttcgagctttgagttcgagttgggcggctaggcaacgtgaaccaacgtaattaaggtgtttagatgtcaagaaaggtttgagaccttaacaatcaagaatggagtttgctggtgagtggatttttgggtcaagaacaagagcaataacacaagctgctgtgtgatgatgaatcttgtcttgcggctaaaggaaatgtttgaggaaagttgtacatatgatcagcaacgtaaagtttatagctttttagaaaccaacttttcgatggttgttcttttttttttgtatgtaatattatagataagcttgtggtatatatgattttaatgtgacattacgaacttgatttataaccatggtaaaattatttaacaaaatatgtaataatagaatgggggaaggggaataaat

SEQ ID NO: 13 - protein, 927 aa, Arabidopsis thaliana
MEARIEGEVEGHSLEYGFSGKRSVEWDLNDWKWNGDLFVATQLNHGSSNSSSTCSDEGNVEIMERRRIEMEKKKKRRAVTVVAMEEDNLKDDDAHRLTLNLGGNNIEGNGVKKTKLGGGIPSRAICCQVDNCGADLSKVKDYHRRHKVCEIHSKATTALVGGIMQRFCQQCSRFHVLEEFDEGKRSCRRRLAGHNKRRRKANPDTIGNGTSMSDDQTSNYMLITLLKILSNIHSNQSDQTGDQDLLSHLLKSLVSQAGEHIGRNLVGLLQGGGGLQASQNIGNLSALLSLEQAPREDIKHHSVSETPWQEVYANSAQERVAPDRSEKQVKVNDFDLNDIYIDSDDTTDIERSSPPPTNPATSSLDYHQDSRQSSPPQTSRRNSDSASDQSPSSSSGDAQSRTDRIVFKLFGKEPNDFPVALRGQILNWLAHTPTDMESYIRPGCIVLTIYLRQDEASWEELCCDLSFSLRRLLDLSDDPLWTDGWLYLRVQNQLAFAFNGQVVLDTSLPLRSHDYSQIITVRPLAVTKKAQFTVKGINLRRPGTRLLCTVEGTHLVQEATQGGMEERDDLKENNEIDFVNFSCEMPIASGRGFMEIEDQGGLSSSFFPFIVSEDEDICSEIRRLESTLEFTGTDSAMQAMDFIHEIGWLLHRSELKSRLAASDHNPEDLFSLIRFKFLIEFSMDREWCCVMKKLLNILFEEGTVDPSPDAALSELCLLHRAVRKNSKPMVEMLLRFSPKKKNQTLAGLFRPDAAGPGGLTPLHIAAGKDGSEDVLDALTEDPGMTGIQAWKNSRDNTGFTPEDYARLRGHFSYIHLVQRKLSRKPIAKEHVVVNIPESFNIEHKQEKRSPMDSSSLEITQINQCKLCDHKRVFVTTHHKSVAYRPAMLSMVAIAAVCVCVALLFKSCPEVLYVFQPFRWELLEYGTS

SEQ ID NO: 14 - DNA, 3634 bp, Arabidopsis thaliana
  source 1..3634: mol_type = genomic DNA
attttttcttatttaaaaaggcgaaatcgagtgagacacgaaatatggggaagtggtccccaccattatttccatttttttatttcttcttcttcttcttctctgtttctctttctgtgtcgaaatgaagatgctttaaatctaacttcttttttttgtttgggcactataaaaccatatccttaccgctaccaaacgcttctcctttaatcatcatcatcatcattttatctccgtcccttcatgttccgttaccgttaacggctgtttttttttaatctccggtgaccgttctttttctgattccaggtgttgccagctgtttttttttttgggagatattttttttaattccttttaaagttagttataagtcagttttgtctgcttggattttcatttataaaacaaggtggaattaggtttcttccgatagagaaaggggcttgattttggacaaaggcacgtgtcttagtcttttattcaagctccgatctataagttatgatttgggtttggttttgagttctccaattttagtctcatctctgtggttgttgtaactgggtttgtgatggaagctagaattgaaggtgaagtagaaggtcacagtttagaatacgggtttagtggtaaaaggagtgtagagtgggatttgaatgattggaaatggaatggtgatctcttcgttgctacacagctgaatcacggctcatccaacagctcttctacgtgctctgatgaaggaaatgttgaaatcatggagagaaggaggatagagatggagaagaagaagaaaagaagagctgttactgtggtagcaatggaagaagataacttgaaagatgatgatgctcatagacttactctgaatcttggtggtaataatattgaggggaatggtgtgaaaaagacgaaacttggaggagggattccgagtcgggcgatttgttgtcaggtagacaactgtggagctgatttaagcaaagttaaggattatcatagacgtcataaggtctgtgagattcattctaaagctactactgcacttgttggaggaattatgcagcggttttgtcagcaatgtagtaggtttcatgtgcttgaagagtttgatgagggaaagagaagttgccgtagacgtttggctgggcataataagcgtagaagaaaagcaaatcctgatactataggcaatgggacttctatgagtgatgatcaaacaagcaattatatgttgattactctcttgaagatactctccaatattcattcgaatcagtcggatcaaacgggtgatcaggatctactgtctcatcttctaaagagccttgtaagccaagctggtgaacatatagggaggaatttagttgggcttctacagggtggaggaggactccaggcttctcaaaatattggaaacttatccgctttgctctcactcgagcaagcccctcgagaggatataaaacatcattcagtgtctgaaacgccttggcaagaagtgtatgccaatagtgctcaagagagagttgccccggataggtccgagaaacaagtcaaagtgaatgattttgatttgaatgacatctacatagactcagatgataccacagatatagagagatcatcacctcctccgacaaatccagctactagttctcttgattatcatcaagactcgcgtcagtctagtccacctcaaactagtaggaggaattcagattcagcttctgaccaatcaccttcaagttccagtggagatgcacagagccgtactgatcggattgtgttcaaactctttgggaaagagccgaatgattttccagttgccttacgaggacagattcttaactggttagcgcatactccaactgatatggagagctacattagacctggttgtatcgttttgaccatttatcttcgtcaagatgaagcttcttgggaagaactttgttgtgatctgagtttcagcttgaggaggcttctagatctttcagatgatcctttatggactgatgggtggctttatcttagggtgcaaaaccaactcgcatttgcgtttaatggtcaggttgttcttgacacatctttacctctgagaagccatgattatagccaaatcattactgttagaccacttgctgtaacaaagaaagctcaatttacagtaaaaggcatcaatctccgtcgacctggcacaaggttactttgtactgttgaaggaacacacttggttcaggaagcaacacaaggagggatggaggagagagatgatctaaaggagaataatgagattgattttgtcaatttttcctgtgagatgcctattgcaagtggtcgaggtttcatggaaattgaagaccaaggtgggctaagcagtagtttcttccctttcatagtatctgaagacgaagatatttgttctgaaatccgaagactcgaaagcacattagagtttactggaactgattctgcaatgcaagcaatggatttcatccacgaaatcggttggcttctacacagaagtgaactcaagtcaagacttgcagcatcagatcataatccagaggatctgttttctttaatacggtttaagtttctaatagagttctcaatggaccgggaatggtgctgtgtgatgaagaagttattgaatattctattcgaagaaggcactgttgatccatctcctgatgctgcgttatcagaactgtgtcttcttcacagagccgtgaggaaaaattcaaaacccatggttgagatgcttcttagattcagtcccaagaagaagaatcagacattagccggtttgtttagacccgatgcagctggtccaggcggtttaacaccgcttcacattgcagctggtaaagacggttcagaagatgtgttggatgccctgactgaggatcctggaatgactggtattcaagcctggaaaaattcccgggacaataccggattcacaccagaagactatgcacgtttacgaggtcacttctcgtatattcatctggtgcaacggaaactcagtcggaaaccaatcgccaaagaacacgtggtggtcaacattcctgagtctttcaacattgagcacaaacaagaaaagaggagtccaatggattcttcaagcttggagataacacagataaaccaatgtaagctctgtgatcacaaacgtgtgtttgtcacaacgcaccacaagtctgttgcctacaggccagcaatgctttcgatggtagcgatagcagcggtttgcgtttgtgtcgctcttttgttcaagagttgcccggaagtgctatatgtctttcagccattcaggtgggaattacttgagtatggaacaagctagtaaaacttccctataccagacattgaaaaagaatcttctcaatagaagatgtgttggtgtgaaatctacgagtgtaattttatgtaacgtttaattatttttactagaatatgtatcattcagctttacaagattatatgtaatgtagctttatctgatgttaaacccccgaatttgtataactacgtttttgtgtgtttgttacattttgtactttgtctttgatggcaaatcttgaagtgagtgagaataacaacaaatcacataaaacaccaa

SEQ ID NO: 15 - protein, 256 aa, Arabidopsis thaliana
MGRGRIEIKKIENINSRQVTFSKRRNGLIKKAKELSILCDAEVALIIFSSTGKIYDFSSVCMEQILSRYGYTTASTEHKQQREHQLLICASHGNEAVLRNDDSMKGELERLQLAIERLKGKELEGMSFPDLISLENQLNESLHSVKDQKTQILLNQIERSRIQEKKALEENQILRKQVEMLGRGSGPKVLNERPQDSSPEADPESSSSEEDENDNEEHHSDTSLQLGLSSTGYCTKRKKPKIELVCDNSGSQVASD

SEQ ID NO: 16 - DNA, 1452 bp, Arabidopsis thaliana
  source 1..1452: mol_type = genomic DNA
gaaaaaccctagaagataaaaaaaaagggaggcgatacacgaaatagagaagagatccaaaaccaaaacaagggcgttataaatgggaagaaagttccaaaagaagaaaacagccactcacaccaagcaaagcaaattagcaaagccaccaaatatggaaaattgctacttatagtaactcccatctttttatataataccctaattttggtcctaattttccttccttagcccctttcccaatatttgatcaatttctaaagaaaaccccttcctctactagtcctcctcctatatatacaaaatcttaagaaatctctctacttgtttcctctgttatcataatctcttctctctatatctcttctcttcttcttttaccctgttttttttttcattccacagagcccaggttgattgattttgttattcagagatatggggagaggaaggattgagattaagaagattgagaatatcaacagtcgtcaagtcactttctctaagagacgaaacggtttgatcaagaaggctaaagagctttcgattctctgtgacgccgaggttgctcttatcatcttctccagcaccggcaagatttacgatttctccagcgtctgtatggagcaaattctttctagatatggatacactactgcgtccactgagcataaacaacaaagagaacaccaacttctaatttgtgcttcacatggaaatgaagctgtgttgcgaaatgatgattctatgaagggggaacttgaaagattacagcttgcaattgagagacttaagggtaaggagcttgaaggtatgagtttcccggatcttatttctcttgaaaaccagttgaacgagagcttgcatagtgtcaaggatcaaaagacacaaatcctgctcaaccagattgagagatccaggatacaggagaaaaaagcattggaagaaaaccaaatcttgcgcaaacaggttgagatgttggggagaggttcaggaccaaaagtgttgaatgaaaggcctcaagattctagcccagaagccgatcccgagagctcttcatcagaagaggatgagaatgacaacgaggagcaccattccgacacttccttgcagttggggttgtcgtcgacggggtattgcacaaagagaaagaagccgaagatcgaactggtctgcgataactctgggagtcaagtggcttctgattgatggaatcgattatttttctaattctggttgtttaggggtctctatgtgtcttcttgtttctggctgttcttttgctttatttcatctcaagtagagttttcttaatgtttaggtggaacatttttccataatcaagaagggatttgatcaatcaataacattagattttcttagttaaagacttaaagttgcccacacaccacaccatatgtgattatgatgaatttacattttataaacagg

SEQ ID NO: 17 - DNA, 274 bp, synthetic construct
  source 1..274: mol_type = other DNA; note = Insertion flanking sequence for MYBS2 mutant
tttgtttatgccacacaagtatacagaaccctacttctaaattgcttctccactggattttaatctcctaactaagcaggaaagacatttctaaacacccgtacaacgtatttgaaaatgaacctcgtgatttcttgtccaggaggttcttctatcaccgccaaaatccaatttctcgaaaaagcacatgtaaaccaccaaattatcaagatgacacgggagtagatcaagataatggcgaaaacacaagtttataaaaatggtgtaagaatct

SEQ ID NO: 18 - DNA, 95 bp, synthetic construct
  source 1..95: mol_type = other DNA; note = Insertion flanking sequence for ATHB25 mutant
aaggatgttagctttagctgaggggattggatggagaagtcagagacaagacgatgaagtgattcagagattttgtcaggatatattgtggtgta

SEQ ID NO: 19 - DNA, 95 bp, synthetic construct; identical to SEQ ID NO: 18
  source 1..95: mol_type = other DNA; note = Insertion flanking sequence for CESTA1 mutant

SEQ ID NO: 20 - DNA, 79 bp, synthetic construct
  source 1..79: mol_type = other DNA; note = Insertion flanking sequence for bHLH093 mutant
gagacgacggagaaaacgacttaacgatcgtcagattgacgcttagacaacttaatgttagcagatcggaagagcggtt